(19) United States
(12) Patent Application Publication
(10) Pub. No.: US 2025/0295290 A1
(43) Pub. Date: Sep. 25, 2025

(54) VISION-BASED ANATOMICAL FEATURE LOCALIZATION

(71) Applicant: Auris Health, Inc., Santa Clara, CA (US)

(72) Inventors: Austin Jun Shin, Mountain View, CA (US); Elif Ayvali, Redwood City, CA (US); Menglong Ye, Mountain View, CA (US); Sean Paul Walker, Fremont, CA (US)

(73) Assignee: Auris Health, Inc., Santa Clara, CA (US)

(21) Appl. No.: 19/083,362

(22) Filed: Mar. 18, 2025

Related U.S. Application Data
(60) Provisional application No. 63/567,679, filed on Mar. 20, 2024.

(51) Int. Cl. A61B 1/00; A61B 1/307; A61B 17/34

(52) U.S. Cl. CPC A61B 1/0005; A61B 1/00006; A61B 1/000096; A61B 1/00149; A61B 1/307; A61B 17/3403

(57) ABSTRACT

A robotic system includes a robotic manipulator configured to manipulate an endoscope having a camera associated therewith and control circuitry configured communicatively coupled to the robotic manipulator. The control circuitry can be configured to receive an image depicting a field-of-view (FOV) of the camera associated with the instrument, detect an anatomical feature in the image, display a graphical interface that includes the image and a visual overlay indicating a location of the anatomical feature in the image, and track the anatomical feature based at least in part on determining that the anatomical feature is a target anatomical feature.

### CROSS-REFERENCE TO RELATED APPLICATIONS

This application claims priority and benefit under 35 U.S.C. § 119 (e) to U.S. Provisional Patent Application No. 63/567,679, filed Mar. 20, 2024, which is incorporated herein by reference in its entirety.

### TECHNICAL FIELD

This disclosure relates generally to medical systems, and specifically to vision-based anatomical feature localization

### BACKGROUND

The present disclosure relates to the field of medical procedures. Various medical procedures involve the use of one or more scope and/or percutaneous access instruments. The improper positioning or advancement of such devices can result in certain physiological and procedural complications.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a robotic system including a robotic manipulator configured to manipulate an instrument having a camera associated therewith and control circuitry communicatively coupled to the robotic manipulator. The control circuitry is configured to receive an image depicting a field-of-view (FOV) of the camera associated with the instrument; detect an anatomical feature in the image; display a graphical interface that includes the image and a visual overlay indicating a location of the anatomical feature in the image; determine whether the anatomical feature is a target anatomical feature based at least in part on a position of the visual overlay relative to the image; and track the anatomical feature based at least in part on determining that the anatomical feature is a target anatomical feature.

Another innovative aspect of the subject matter of this disclosure can be implemented in a method of target localization. The method includes steps of receiving an image depicting a field-of-view (FOV) of a camera associated with an instrument coupled to a robotic manipulator; detecting an anatomical feature in the image; displaying a graphical interface that includes the image and a visual overlay identifying the anatomical feature in the image; determining whether the anatomical feature is a target anatomical feature based at least in part on a position of the visual overlay relative to the image; and tracking the anatomical feature based at least in part on determining that the anatomical feature is a target anatomical feature.

Another innovative aspect of the subject matter of this disclosure can be implemented in a controller for a robotic system, including a processing system and a memory. The memory stores instructions that, when executed by the processing system, cause the controller to receive an image depicting a field-of-view (FOV) of a camera associated with an instrument coupled to a robotic manipulator; detect an anatomical feature in the image; display a graphical interface that includes the image and a visual overlay identifying the anatomical feature in the image; determine whether the anatomical feature is a target anatomical feature based at least in part on a position of the visual overlay relative to the image; and track the anatomical feature based at least in part on determining that the anatomical feature is a target anatomical feature.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an embodiment of a robotic medical system in accordance with one or more embodiments.

FIG. 2 illustrates a robotic control system in accordance with one or more embodiments.

FIG. 3 illustrates a robotic system in accordance with one or more embodiments.

FIG. 4 illustrates a robotically-controllable endoscope in accordance with one or more embodiments.

FIG. 5 illustrates a robotic instrument feeder in accordance with one or more embodiments.

FIGS. 6A and 6B provide a flow diagram illustrating a process for performing guided percutaneous nephrolithotomy in accordance with one or more embodiments.

FIGS. 7, 8, and 9 show images of anatomy and instrumentation corresponding to various blocks, states, and/or operations associated with the process of FIGS. 6A and 6B, in accordance with one or more embodiments.

FIG. 10 is a flow diagram illustrating a process for localizing a target anatomical feature in accordance with one or more embodiments.

FIG. 11 shows a scope device disposed within a target calyx for target localization in accordance with one or more embodiments.

FIG. 12 illustrates an image-based anatomical feature tracking architecture in accordance with one or more embodiments.

FIGS. 13A, 13B, 13C, and 13D show camera images of an anatomical site in accordance with one or more embodiments.

FIG. 14 shows a camera image of an anatomical site, the image including bounding-box features in accordance with one or more embodiments.

FIG. 15 illustrates a robotic medical system arranged to facilitate navigation of a scope within a patient in accordance with one or more embodiments.

FIG. 16 illustrates a scope camera view/window including endoscope positioning guidance features in accordance with one or more embodiments.

FIG. 17 shows renal anatomy with a ureteroscope parked at various positions in accordance with one or more embodiments.

FIG. 18 illustrates a scope camera view/window including anatomical feature location guidance features in accordance with one or more embodiments.

FIG. 19 illustrate a scope camera view/window including anatomical feature tracking features in accordance with one or more embodiments.

FIG. 20 illustrates a three-dimensional, image-based position estimation framework in accordance with one or more embodiments.

FIG. 21 is a flow diagram illustrating a process for triangulating a target anatomical feature in accordance with one or more embodiments.

FIG. 22 illustrates a graphical interface representing third-person-perspective endoscope positioning guidance in accordance with one or more embodiments.

FIG. 23 is a flow diagram illustrating a process for autonomous triangulation of a target anatomical feature in accordance with one or more embodiments.

FIG. 24 illustrates a scope camera view/window including instrumentation tracking features in accordance with one or more embodiments.

FIG. 25 illustrates a scope camera view/window including endoscope positioning guidance features in accordance with one or more embodiments.

FIG. 26 is a block diagram of an anatomical feature tracking framework in accordance with one or more embodiments.

FIG. 27 shows a block diagram of an example controller for a robotic system, according to some implementations.

FIG. 28 shows an illustrative flowchart depicting an example target localization operation, according to some implementations.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

### Overview

The present disclosure relates to systems, devices, and methods for localizing and targeting anatomical features to aid in certain medical procedures. Although certain aspects of the present disclosure are described in detail herein in the context of renal, urological, and/or nephrological procedures, such as kidney stone removal/treatment procedures, it should be understood that such context is provided for convenience and clarity, and anatomical feature localizing and targeting concepts disclosed herein are applicable to any suitable medical procedures.

In accordance with certain surgical procedures disclosed herein, endoscopes (e.g., ureteroscopes) can be equipped with one or more position sensors, wherein the position of the sensor(s) is used as a target for percutaneous access, such as for PCNL. For example, an electromagnetic-sensor-equipped ureteroscope and/or an electromagnetic-sensor-equipped percutaneous access needle may be used to guide the percutaneous renal access for kidney stone removal and/or the like. In such procedures, the surgeon/physician can drive the ureteroscope to a target calyx of the kidney and use an electromagnetic sensor (e.g., beacon) associated with a distal end/tip of the ureteroscope as the percutaneous access target for the needle. Generally, the efficacy of percutaneous axis to a target calyx can depend at least in part on where the physician positions/parks the ureteroscope with respect to, for example, the position and/or heading of the target calyx and/or papilla through which percutaneous access may be made to the target calyx. For some procedures in which the distal end/tip of the ureteroscope is used as the percutaneous access target, it may be desirable for the distal tip of the ureteroscope to be as close as possible to the papilla/calyx interface during percutaneous access/approximation.

Robotic-assisted percutaneous procedures can be implemented in connection with various medical procedures, such as kidney stone removal procedures, wherein robotic tools can enable a physician/urologist to perform endoscopic (e.g., ureteroscopy) target access as well as percutaneous access/treatment. Advantageously, aspects of the present disclosure relate to real-time target tracking/localization in medical procedures, which may be utilized by the operating physician to direct a percutaneous-access instrument (e.g., needle or other rigid tool) and/or to guide robotic instrumentation, such as by adjusting endoscope position and/or alignment automatically in response to such real-time target-tracking information. To facilitate such functionality, embodiments of the present disclosure may advantageously provide mechanisms for anatomical feature target localizing, tracking, and/or three-dimensional position estimation using scope camera image data to assist physicians to achieve relatively efficient and accurate percutaneous access for various surgical operations, such as nephroscopy. In determining the initial placement of a percutaneous access target, incorporating visual information from scope camera data can provide a variety of benefits. For example, information extracted from visual data can allow a physician to determine if the features in view are the desired target feature(s) for percutaneous access path targeting. With respect to nephrolithotomy applications, use of scope camera image data can resolve ambiguity about whether infundibular tissue or the target papilla has been contacted or whether the papilla in view is the desired target papilla.

### Robotic Surgical Systems

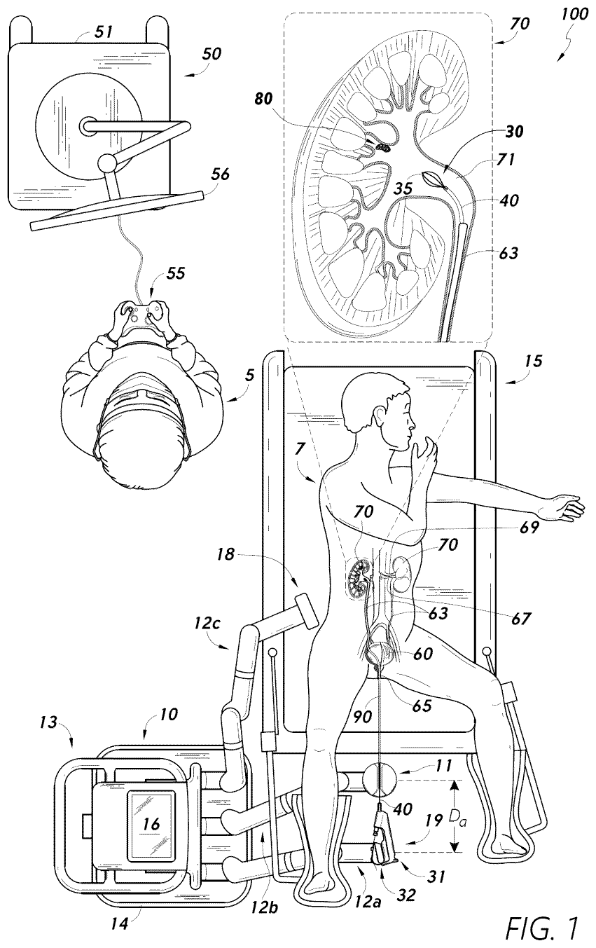

FIG. 1 illustrates an example medical system 100 for performing various medical procedures in accordance with aspects of the present disclosure. The medical system 100 may be used for, for example, endoscopic (e.g., ureteroscopic) procedures. As referenced and described above, certain ureteroscopic procedures involve the treatment/removal of kidney stones. In some implementations, kidney stone treatment can benefit from the assistance of certain robotic technologies/devices. Robotic medical solutions can provide relatively higher precision, superior control, and/or superior hand-eye coordination with respect to certain instruments compared to strictly-manual procedures. For example, robotic-assisted ureteroscopic access to the kidney in accordance with some procedures can advantageously enable a urologist to articulate a ureteroscope using robotically-controlled gears/drives coupled to a handle/base portion of the ureteroscope. Although the system 100 of FIG. 1 is presented in the context of a ureteroscopic procedure, it should be understood that the principles disclosed herein may be implemented in any type of endoscopic procedure.

The medical system 100 includes a robotic system 10 (e.g., mobile robotic cart) configured to engage with and/or control a medical instrument 19 (e.g., endoscope/ureteroscope) including a proximal handle/base 31 and a shaft 40 coupled to the handle 31 at a proximal portion thereof to perform a direct-entry procedure on a patient 7. The term “direct-entry” is used herein according to its broad and ordinary meaning and may refer to any entry of instrumentation through a natural or artificial opening in a patient's body. For example, with reference to FIG. 1, the direct entry of the scope/shaft 40 into the urinary tract of the patient 7 may be made through the urethra 65.

It should be understood that the direct-entry instrument 19 may be any type of shaft-based medical instrument, including an endoscope (such as a ureteroscope), catheter (such as a steerable or non-steerable catheter), nephroscope, laparoscope, or other type of medical instrument. Embodiments of the present disclosure relating to ureteroscopic procedures for removal of kidney stones through a ureteral access sheath (e.g., the ureteral access sheath 90) are also applicable to solutions for removal of objects through percutaneous access, such as through a percutaneous access sheath. For example, instrument(s) may access the kidney percutaneously through a percutaneous access sheath to capture and remove kidney stones. The term “percutaneous access” is used herein according to its broad and ordinary meaning and may refer to entry, such as by puncture and/or minor incision, of instrumentation through the skin of a patient and any other body layers necessary to reach a target anatomical location associated with a procedure (e.g., the calyx network of the kidney 70).

The medical system 100 includes a control system 50 configured to interface with the robotic system 10, provide information regarding the procedure, and/or perform a variety of other operations. For example, the control system 50 can include one or more display(s) 56 configured to present certain information to assist the physician 5 and/or other technician(s) or individual(s). The medical system 100 can include a table 15 configured to hold the patient 7. The system 100 may further include an electromagnetic (EM) field generator 18, which may be held by one or more of the robotic arms 12 of the robotic system 10 or may be a stand-alone device and/or mounted to the table 15. Although the various robotic arms 12 are shown in various positions and coupled to various tools/devices, it should be understood that such configurations are shown for convenience and illustration purposes, and such robotic arms may have different configurations over time and/or at different points during a medical procedure. Furthermore, the robotic arms 12 may be coupled to different devices/instruments than shown in FIG. 1.

Articulation of the shaft 40 may be controlled robotically, such as through operation of an end effector associated with the robot arm 12a, wherein such operation may be controlled by the control system 50 and/or robotic system 10. The term “end effector” is used herein according to its broad and ordinary meaning and may refer to any type of robotic manipulator device, component, and/or assembly. In implementations in which an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. In some contexts, the combination of a robotic end effector and adapter may be referred to as an instrument manipulator assembly, wherein such assembly may or may not also include a medical instrument (or instrument handle/base) physically coupled to the adapter and/or end effector. The terms “robotic manipulator” and “robotic manipulator assembly” are used according to their broad and ordinary meanings, and may refer to a robotic end effector and/or sterile adapter or other adapter component coupled to the end effector, either collectively or individually. For example, the terms “robotic manipulator” and “robotic manipulator assembly” may refer to an instrument device manipulator (IDM) including one or more drive outputs, whether embodied in a robotic end effector, sterile adapter, and/or other component(s). The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

In an example use case, if the patient 7 has a kidney stone (or stone fragment) 80 located in a kidney 70, the physician 5 may perform a procedure to remove the stone 80 through the urinary tract (63, 60, 65). In some embodiments, the physician 5 can interact with the control system 50 and/or the robotic system 10 to cause/control the robotic system 10 to advance and navigate the medical instrument shaft 40 (e.g., a scope) from the urethra 65, through the bladder 60, up the ureter 63, and into the renal pelvis 71 and/or calyx network of the kidney 70 where the stone 80 is located. The control system 50 can provide information via the display(s) 56 that is associated with the medical instrument 40, such as real-time endoscopic images captured therewith, and/or other instruments of the system 100, to assist the physician 5 in navigating/controlling such instrumentation.

With further reference to the medical system 100, the medical instrument shaft 40 (e.g., scope, directly-entry instrument, etc.) can be advanced into the kidney 70 through the urinary tract. Specifically, a ureteral access sheath 90 may be disposed within the urinary tract to an area near the kidney 70. The shaft 40 may be passed through the ureteral access sheath 90 to gain access to the internal anatomy of the kidney 70, as shown. The distal portion of the scope/shaft 40 deployed from the sheath 90 may be articulatable to allow the physician 5 to use inputs of the control device 55 to cause the robotic system 10 to articulate the shaft 40 towards the target kidney stone. Once at the site of the kidney stone 80 (e.g., within a target calyx 75 of the kidney 70 through which the stone 80 is accessible), the medical instrument 19 and/or shaft 40 thereof can be used to channel/direct the basketing device 30 to the target location. Once the stone 80 has been captured in the distal basket portion 35 of the basketing device/assembly 30, the utilized ureteral access path may be used to extract the kidney stone 80 from the patient 7. Advancement and retraction of the scope shaft 40 can be implemented by an instrument feeder 11, which may be coupled to an end effector actuator, as shown.

The various scope/shaft-type instruments disclosed herein, such as the shaft 40 of the system 100, can be configured to navigate within the human anatomy, such as within a natural orifice or lumen of the human anatomy. The terms “scope” and “endoscope” are used herein according to their broad and ordinary meanings, and may refer to any type of elongate (e.g., shaft-type) medical instrument having image generating, viewing, and/or capturing functionality and being configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise an at least partially rigid and/or flexible tube, and may be dimensioned to be passed within an outer sheath, catheter, introducer, or other lumen-type device, or may be used without such devices.

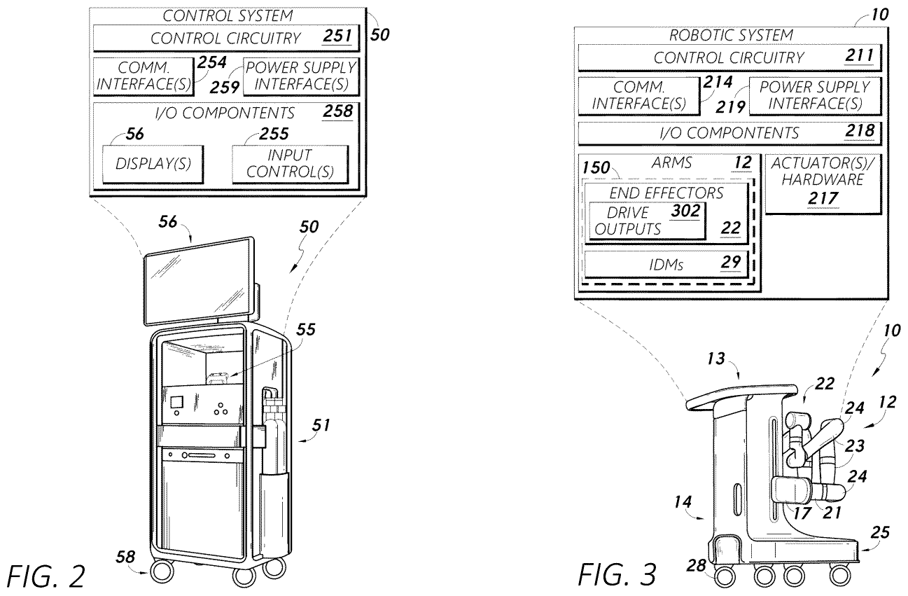

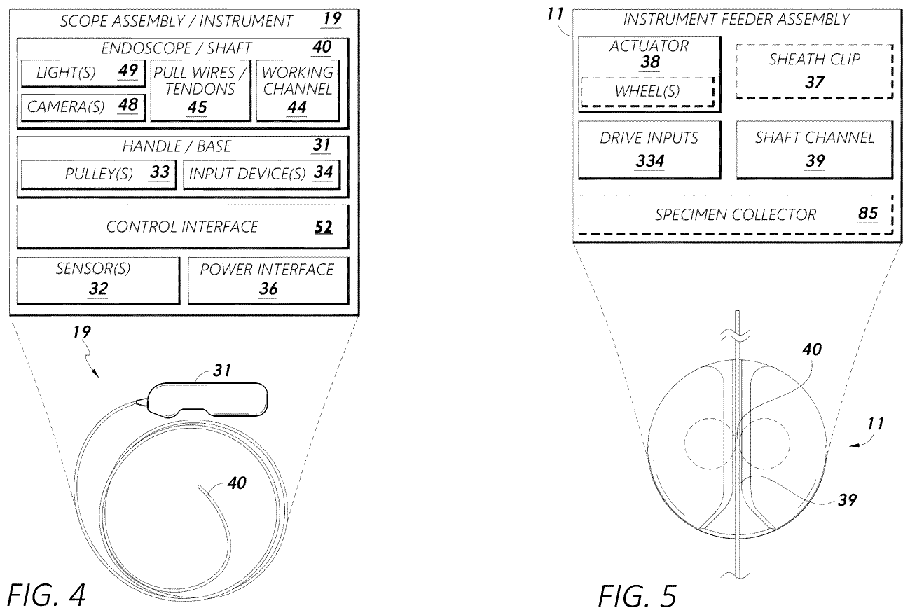

FIG. 2 shows an example embodiment of a control system 50 of any system disclosed herein. FIG. 3 shows an example robotic system 10 of any system disclosed herein. FIG. 4 shows a robotically-controllable endoscope 19 of any system disclosed herein. FIG. 5 shows a robotic instrument feeder 11 of any system disclosed herein.

With reference to FIGS. 2-5, the control system 50 can be coupled to the robotic system 10 and operate in cooperation therewith to perform a medical procedure. For example, the control system 50 can communicate with the robotic system 10 via a wireless connection or a wired connection (e.g., to control the robotic system 10). Further, in some embodiments, the control system 50 can communicate with the robotic system 10 to receive position data therefrom relating to the position of the distal end of the scope 40. Such positional data relating to the position of the scope 40 may be derived using one or more electromagnetic sensors associated with the respective components, scope image processing functionality, and/or based at least in part on robotic system data (e.g., arm position data, known parameters/dimensions of the various system components, etc.).

The robotic system 10 can be arranged in a variety of ways depending on the particular procedure. The robotic system 10 can include one or more robotic arms 12 configured to engage with and/or control, for example, the scope 40 to perform one or more aspects of a procedure. As shown, each robotic arm 12 can include multiple arm segments 23 coupled to joints 24, which can provide multiple degrees of movement/freedom. When the robotic system 10 is properly positioned, the scope 40 can be inserted into a patient robotically using the robotic arms 12, manually by the physician 5, or a combination thereof. The instrument feeder 11 can be attached to the distal end effector 22 of one of the arms 12 to facilitate robotic control/advancement of the scope 40. Another arm 12 may have associated therewith an instrument base/handle 31, wherein the scope 40 is physically coupled to the handle 31 at a proximal end of the scope 40. The scope 40 may include one or more working channels 44 through which additional tools, such as lithotripters, basketing devices, forceps, etc., can be introduced into the treatment site.

The robotic system 10 may be configured to receive control signals from the control system 50 to perform certain operations, such as to position one or more of the robotic arms 12 in a particular manner, manipulate (e.g., advance, articulate) the scope 40, and so on. In response, the robotic system 10 can control, using certain control circuitry 211, actuators 217, and/or other components of the robotic system 10, to perform the operations. For example, the control circuitry 211 may control articulation of the shaft/scope 40 by actuating drive output(s) of the end effector 22 coupled to the instrument handle 31. In some embodiments, the robotic system 10 and/or control system 50 is/are configured to receive images and/or image data from the scope 40 representing internal anatomy of a patient and/or portions of the access sheath or other device components.

The robotic system 10 generally includes an elongated support structure 14 (also referred to as a “column”), a robotic system base 25, and a console 13 at the top of the column 14. The column 14 may include one or more arm supports 17 (also referred to as a “carriage”) for supporting the deployment of the one or more robotic arms 12 (three shown in FIGS. 1 and 2). The arm support 17 may include individually configurable arm mounts that rotate along a perpendicular axis to adjust the base of the robotic arms 12 for desired positioning relative to the patient.

The arm support 17 may be configured to vertically translate along the column 14. Vertical translation of the arm support 17 allows the robotic system 10 to adjust the reach of the robotic arms 12 to meet a variety of table heights, patient sizes, and physician preferences. Similarly, the individually configurable arm mounts on the arm support 17 can allow the robotic arm base 21 of robotic arms 12 to be angled in a variety of configurations.

The robotic arms 12 may generally comprise robotic arm bases 21 and end effectors 22, separated by a series of linking arm segments 23 that are connected by a series of joints 24, each joint 24 comprising one or more independent actuators 217. Each actuator may comprise an independently controllable motor. Each independently controllable joint 24 can provide or represent an independent degree of freedom available to the robotic arm.

The robotic system base 25 balances the weight of the column 14, arm support 17, and arms 12 over the floor. Accordingly, the robotic system base 25 may house certain relatively heavier components, such as electronics, motors, power supply, as well as components that selectively enable movement or immobilize the robotic system. For example, the robotic system base 25 can include wheel-shaped casters 28 that allow for the robotic system to easily move around the operating room prior to a procedure.

Positioned at the upper end of column 14, the console 13 can provide input/output (I/O) components 218, such as a user interface for receiving user input and a display screen 16 (or a dual-purpose device such as, for example, a touchscreen) to provide the physician/user 5 with both pre-operative and intra-operative data. Potential pre-operative data on the console/display 16 or display 56 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse.

The end effector 22 of each of the robotic arms 12 may comprise, or be configured to have coupled thereto, an instrument device manipulator (IDM) (e.g., instrument base/handle) 29, which may be attached using a sterile adapter component in some instances. The combination of the end effector 22 and associated IDM, as well as any intervening mechanics or couplings (e.g., sterile adapter), can be referred to as a manipulator assembly. In some embodiments, the IDM 29 can be removed and replaced with a different type of IDM, for example, a first type of IDM/instrument may be configured to manipulate an endoscope/shaft, while a second type of IDM/instrument may be associated with the shaft 40 (e.g., coupled to a proximal portion thereof) and configured to articulate the shaft. An IDM can provide power 219 and control/communication 214 interfaces. For example, the interfaces can include connectors to transfer pneumatic pressure, electrical power, electrical signals, and/or optical signals from the robotic arm 12 to the IDM 29. The IDMs 29 may be configured to manipulate medical instruments (e.g., surgical tools/instruments), such as the scope 40, using techniques including, for example, direct drives, harmonic drives, geared drives, belts and pulleys, magnetic drives, and the like. In some embodiments, the device manipulators 29 can be attached to respective ones of the robotic arms 12.

As referenced above, the robotic system 10 can include certain control circuitry 211, and further the control system 50 can include control circuitry 251. Any reference herein to control circuitry may refer to circuitry embodied in a robotic system, a control system, or any other component of a medical system. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211,251 may be entirely locally maintained/disposed or may be remotely located at least in part (e.g., communicatively coupled indirectly via a local area network and/or a wide area network).

With respect to the robotic system 10, at least a portion of the control circuitry 211 may be integrated with the base 25, column 14, and/or console 13 of the robotic system 10, and/or another system communicatively coupled to the robotic system 10. With respect to the control system 50, at least a portion of the control circuitry 251 may be integrated with the console base 51 and/or display unit 56 of the control system 50. It should be understood that any description herein of functional control circuitry or associated functionality may be understood to be embodied in the robotic system 10, the control system 50, or any combination thereof, and/or at least in part in one or more other local or remote systems/devices, such as control circuitry associated with a handle/base of a shaft-type instrument (e.g., endoscope) in accordance with any of the disclosed embodiments.

The control system 50 can include various I/O components 258 configured to assist the physician or others in performing a medical procedure. For example, the input/output (I/O) components 258 can be configured to allow for user input to control/navigate the scope 40 and/or other robotically controlled instrument. The control system 50 can include one or more display devices 56 to provide various information regarding a procedure. For example, the display(s) 56 can provide information regarding the scope 40. For example, the control system 50 can receive real-time images that are captured by the scope 40 and display the real-time images via the display(s) 56. Additionally, or alternatively, the control system 50 can receive signals (e.g., analog, digital, electrical, acoustic/sonic, pneumatic, tactile, hydraulic, etc.) from a medical monitor and/or a sensor associated with the patient, and the display(s) 56 can present information regarding the health or environment of the patient. Wheels 58 or other mobility means may facilitate movement of the cart within the surgical environment.

The various components of the systems of FIGS. 1-5 can be communicatively coupled to each other over a network, which can include a wireless network and/or a wired network. Example networks include one or more personal area networks (PANs), local area networks (LANs), wide area networks (WANs), Internet area networks (IANs), cellular networks, the Internet, personal area networks (PANs), body area network (BANs), etc. In some embodiments, the various communication interfaces 254 can implement a wireless technology such as Bluetooth, Wi-Fi, near-field communication (NFC), or the like. Furthermore, in some embodiments, the various components of the systems can be connected for data communication, fluid exchange, power exchange 259, and so on via one or more support cables, tubes, or the like.

The control system 50 and/or the robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to the respective control circuitry. In some embodiments, the user may engage the user controls 55 to command robotic shaft articulation, as described herein.

With reference to FIG. 4, the scope assembly 19 includes a handle or base 31 coupled to an endoscope shaft 40. For example, the endoscope (i.e., “scope” or “shaft”) can include an elongate shaft including one or more lights 49 and one or more cameras or other imaging devices 48. The scope 40 can further include one or more working channels 44, which may run a length of the scope 40. The scope assembly 19 can be powered through a power interface 36 and/or controlled through a control interface 52, each or both of which may interface with a robotic arm/component of the robotic system 10. The scope assembly 19 may further comprise one or more sensors 32, such as pressure sensors and/or other force-reading sensors, which may be configured to generate signals indicating forces experienced at/by one or more components of the scope assembly 19.

The scope assembly 19 includes certain mechanisms for causing the shaft 40 to articulate/deflect with respect to an axis thereof. For example, the shaft 40 may have been associated with a proximal portion thereof, one or more drive inputs 34 associated, and/or integrated with one or more pulleys/spools 33 that are configured to tension/untension pull wires 45 of the scope shaft 40 to cause articulation of the shaft 40.

With reference to FIG. 5, the instrument feeder assembly 11 can include a channel 39 dimensioned and/or configured for placement therein of at least a portion of a shaft-type instrument, such as an endoscope or the like. For example, when placing a scope or the like to allow for the instrument feeder 11 to axially drive such instrument, the instrument may be nested at least partially within the channel 39. Although illustrated with a channel 39, in some embodiments, instrument feeder devices and assemblies in accordance with aspects of the present disclosure may not include such a channel. The actuator 38 may comprise a feed-roller in some embodiments, including any number of roller(s)/wheel(s) configured to effect axial movement of a shaft engaged therewith. The actuator(s) 38 can be controlled through engagement with one or more drive inputs 83, which may allow for physical engagement with mechanical components of the instrument feeder 11 that actuate the actuator 38 and/or may directly actuate the actuator 38. The feeder 11 can include a sheath clip 37 for securing an access sheath through with the endoscope passes to the feeder 11.

### Target Localization for Percutaneous Nephrolithotomy

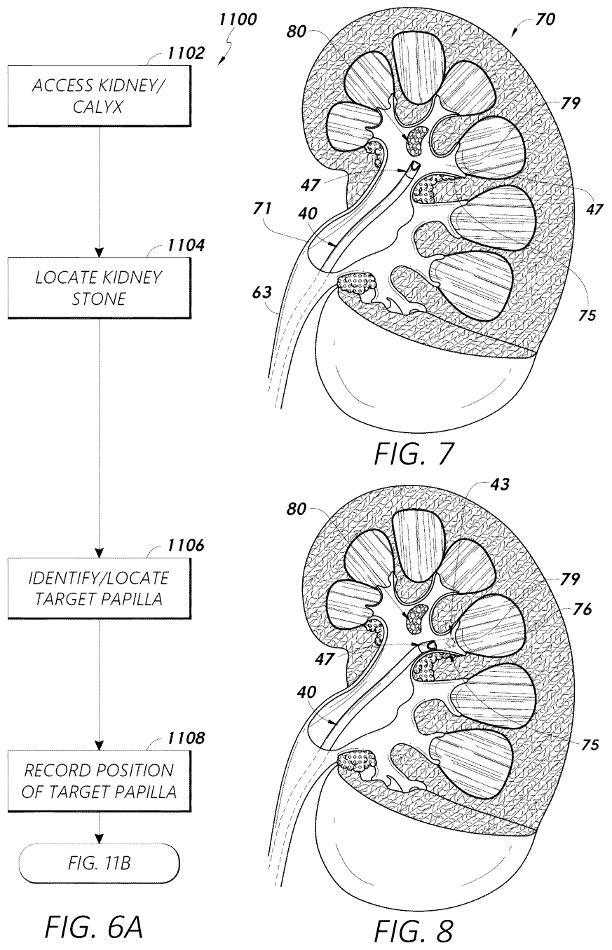

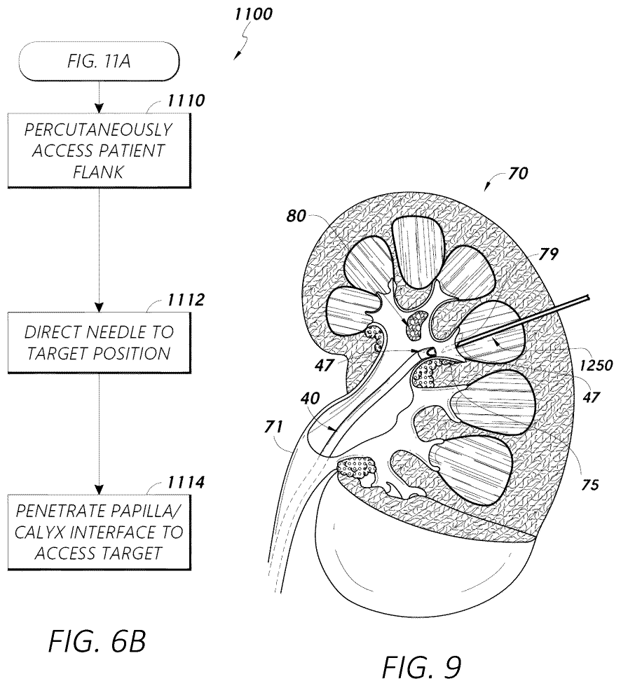

FIGS. 6A and 6B provide a flow diagram illustrating a process 1100 for performing guided percutaneous nephrolithotomy in accordance with one or more embodiments. FIGS. 7, 8, and 9 show images of anatomy and instrumentation corresponding to various blocks, states, and/or operations associated with the process of FIGS. 6A and 6B, respectively, in accordance with one or more embodiments.

For percutaneous nephrolithotomy (PCNL) procedures, access is made into the target calyx through the skin and intervening tissue of the patient using a needle. Generally, access to the calyces of the kidney is through the soft-tissue papilla structures through a needle path that traverses surrounding organs and also allows for a rigid instrument to reach and treat the urinary stone. Failure to select the proper path can cause visceral or pleural injury or inability to complete the intended treatment.

In some procedures, the physician(s) study a patient's preoperative computed tomography (CT) images to determine the location of the urinary stone, the location of surrounding organs and bony structures, and examine the morphometry of the calyces. Such information can help the physicians to create a pre-operative plan for the percutaneous needle path. Intraoperatively, physicians in some procedures use fluoroscopy or ultrasound to guide the alignment and insertion of the needle to the target calyx. However, the resolution and interpretation difficulty associated with such imaging techniques can result in a relatively high degree of difficulty in satisfactorily executing the needle puncture. Embodiments of the present disclosure provides tracking and visualization of target anatomical features, such as papillas and calyces.

With reference to FIGS. 6A and 7, the process 1100 includes percutaneous access to the kidney 70 for kidney stone removal (e.g., PCNL). Such percutaneous access may be desirable for extraction of stones that are sufficiently large that removal via ureteroscope is impractical or undesirable. The processes described herein, although described in the context of ureteroscope, may apply to any other type of surgical procedure utilizing a position sensor (e.g., electromagnetic field sensor) and/or camera to track a target anatomical feature, such as a papilla or urinary stone.

At block 1102, the process 1100 involves accessing the kidney 70 through the ureter 63 of the patient using a scope 40, as described above. In particular, the operation of block 1102 may involve advancing the scope 40 through the ureter 63, past the renal pelvis 71, and into an area at or near one or more calyces 75.

At block 1104, the process 1100 involves locating, using an image-capturing device (e.g. camera) associated with the distal end 47 of the scope 40, a kidney stone 80, for which the patient is to be treated.

At block 1106, the process 1100 involves identifying a target papilla 79 that is exposed within a target calyx 75 through which access to the kidney stone 80 may be achieved percutaneously. Identifying the target papilla 79 may be important for creating a workable tract through which access to the kidney stone 80 can be made via percutaneous access. For example, it may be necessary to determine an angle that is appropriate for access by a relatively rigid percutaneous nephroscope in such a way as to access a calyx (e.g., minor calyx 75) through which the kidney stone 80 can be reached. Generally, minor calyces may be considered relatively small targets. For example, such calyces may be approximately 11-8 mm in diameter. Therefore, precise targeting can be critical in order to effectively extract the kidney stone(s).

The path through which needle/nephroscope access to the target calyx 75 is achieved may advantageously be as straight as possible in order to avoid hitting blood vessels around the renal pyramid 76 associated with the papilla 79 through which the needle/nephroscope may be positioned. Furthermore, the position of various critical anatomy of the patient may necessitate navigation through a constrained window of tissue/anatomy of the patient. For example, the lower pole calyces, below the 12th rib, may provide a suitable access to avoid the pulmonary pleura. Furthermore, the access path may advantageously be medial to the posterior axillary line (e.g. approximately 1 cm below and 1 cm medial to the tip of the 12th rib) to avoid the colon and/or paraspinal muscle. In addition, the access path may advantageously avoid coming within close proximity to the rib to avoid the intercostal nerves. Furthermore, by targeting entry in the area of the axial center of the calyx 75, major arteries and/or other blood vessels can be avoided in some instances.

At block 1108, the process 1100 involves tagging/recording the position of the exposed papilla 79 within the target calyx through which the desired access is to be achieved. For example, position information/data may be represented/identifiable in a three-dimensional space, such as an electromagnetic field space, or a robot space (e.g., coordinate frame). In order to record the position of the papilla 79, the scope 40 may be advanced to physically touch/contact the target papilla 79, as shown by the advanced scope tip 43 in FIG. 8, in connection with which such contact position may be identified and/or otherwise indicated as the target position by the scope 40 and/or operator. In some implementations, an electromagnetic beacon or other sensor device associated with the distal end/tip 47 of the scope 40 may indicate the target position, thereby registering the target position in the electromagnetic field space. After contacting/touching the papilla 79 and recording the position, the end 47 of the scope may be retracted, and the depth of such retraction measured in some manner. In some implementations, the operator may be informed that the distal end 47 of the scope 40 has contacted the papilla 79 by monitoring the camera images generated thereby, which may generally become obstructed/blacked-out when contact is made. In some implementations, a user input device (e.g., pendant) can be used to inform the system that contact has been made with the target anatomical feature.

Certain embodiments of the present disclosure advantageously help to automate and guide physicians through the process for gaining percutaneous to target anatomical features. For example, visual annotations of scope images can be used to guide the insertion of a needle into a patient. Certain embodiments of the present disclosure involve position-sensor-guided percutaneous access to a target treatment site, such as a target location in the kidney. For example, where the scope 40 is fitted with one or more electromagnetic sensors, and the nehproscope access needle further includes one or more electromagnetic sensors, and such sensors are positioned within an electromagnetic field created by a field generator, associated system control circuitry can be configured to detect and track their locations. In some embodiments, the tip of the scope 40 acts as a guiding beacon while the user inserts the percutaneous access needle.

At block 1110, the process 1100 involves percutaneously introducing a medical instrument 1250, such as a needle, into the patient. For example, such access may be made via the flank of the patients in some implementations. At block 1112, the process 1100 involves directing the percutaneously advanced medical instrument 40 towards the target position to ultimately traverse the target papilla 79 and access the target calyx 75 therethrough.

In some embodiments, visual confirmation of the entry of the tip of the needle 1250 into the target calyx 75 may be provided by the camera of the scope 40. For example, the scope 40 may be backed-off from the target position, as described above, to thereby provide a field of view including the papilla 79 within the calyx 75, such that the tip of the needle 1250 may be seen as it protrudes through the surface of the papilla 79.

With the target location recorded, a percutaneously-inserted medical instrument (e.g., the needle 1250) may be directed towards the recorded position. However, where such recorded position is static, anatomical motion occurring after recordation of the target position may result in the target position not accurately reflecting the real-time position associated with the target anatomical feature through which access desired. For example, the act of inserting the needle 1250 into the patient may cause certain anatomy around the target organ (e.g., the kidney 70) and/or the target organ itself to migrate and/or become distorted/misshaped in some manner, thereby causing the target anatomical feature (e.g., papilla 79) to assume a position/shape different than at the time at which the target access position was recorded.

Once needle access has been made to the calyx 75, a larger-diameter sheath device may be exchanged for the needle 1250 to provide a larger port for stone removal. In some implementations, the needle 1250 comprises a stylet and a cannula. With the needle tip advanced into the calyx 75, the stylet may be removed, leaving the cannula to form an open port to the location of the kidney stone. At this point, a nephroscope or any one of a number of other instruments may be introduced into the suction tube to assist in removing the stone 80.

Various aspects of the present disclosure relate to systems, devices, and methods for target (e.g., target anatomical feature) localization in connection with medical procedures. In particular, target localization in accordance with the present disclosure can involve various steps and/or functionality, including recording/tagging a position of a target anatomical feature (e.g., papilla) using an endoscope (e.g., ureteroscope), determining/registering a positional offset/transform between the target anatomical feature and the endoscope (e.g., position sensor associated therewith) for the purpose of determining the position of the target anatomical feature based on the position of an endoscope that is not in physical contact with the target anatomical feature, and/or dynamically updating a target position associated with the target anatomical feature based on electromagnetic sensor and/or camera data associated with the ureteroscope. As described, a static position marker may be registered/recorded to identify a target position associated with a target anatomical feature/landmark. In some embodiments, the present disclosure provides systems, devices, and methods for guiding and/or automating endoscope and/or percutaneous-access instruments based at least in part a static position marker in view of certain target localization techniques. Target localization in accordance with embodiments of the present disclosure can apply to any type of robotic endoscopy procedure.

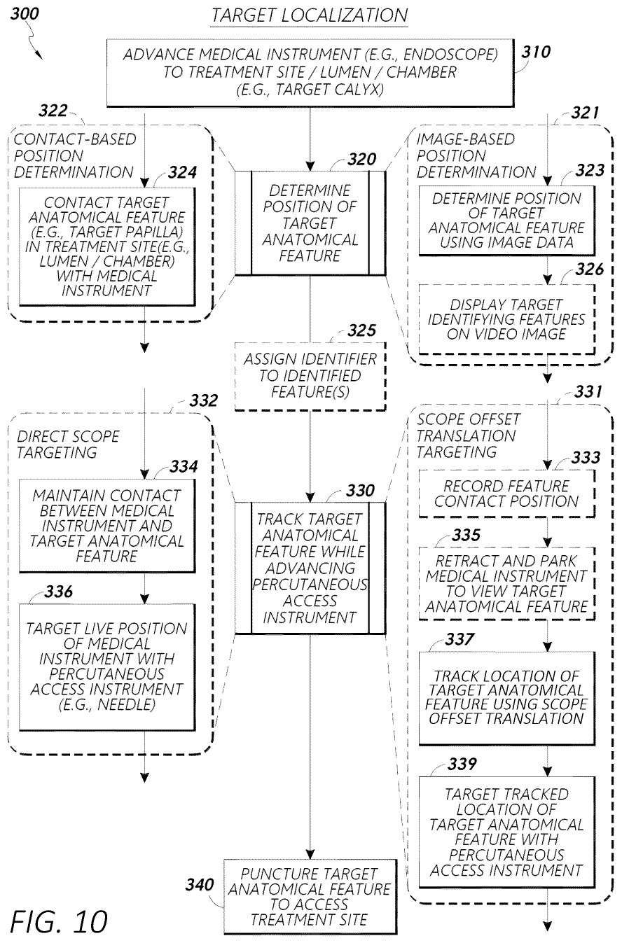

FIG. 10 is a flow diagram illustrating a process 300 for localizing a target anatomical feature in accordance with one or more embodiments. Generally, target localization may be implemented to locate the position of a target anatomical feature (e.g., papilla) with respect to a ureteroscope. The target position may be recorded/saved with respect to any subsystem/data described above, such as an electromagnetic field generator/space, a robot coordinate frame, and/or an anatomical coordinate frame defined by, for example, kidney mapping. At block 310, the process 300 involves advancing a medical instrument, such as a scope (e.g., ureteroscope), to the treatment site, such as a lumen or chamber disposed at least partially within a target organ. For example, the operation of block 310 may involve advancing the medical instrument to a target calyx of the kidney of a patient.

As referenced above, robotic endoscope-guided percutaneous access in accordance with aspects of the present disclosure can utilize target localization technology with respect to the target anatomical feature to guide/determine a percutaneous access path for accessing the target anatomical feature/site. For example, position-tracking mechanisms/sensors associated with the distal end of the medical instruments (e.g., scope) and/or a percutaneous-access instrument (e.g., needle) can be implemented in order to guide the physician/technician in aligning the percutaneous-access instruments with the treatment site (e.g., target calyx). Accurate, real-time target localization/tracking, as enabled by aspects of the present disclosure, can enable relatively precise single-stick access to the treatment site.

At block 320, the process 300 involves determining a position of the target anatomical feature. For example, determining the position of the target anatomical feature can be performed in any suitable or desirable way, such as using an at least partially contact-based position-determination subprocess 322 or an at least partially image-based position-determination subprocess 321, which are described below in connection with blocks 324 and 323/326, respectively.

With respect to certain contact-based position determination processes, at block 322, the process 300 involves contacting the target anatomical feature in the treatment site with the distal end of the medical instrument. For example, the medical instrument may comprise a sensor device, such as an electromagnetic sensor/beacon that may indicate a position of the distal end of the medical instrument, and therefore, with the distal end of the medical instrument disposed against and/or adjacent to the target anatomical feature, such position reading can be relied upon as indicating the present position of the target anatomical feature. Contact-based position determination may not be needed when an image-processing approach is implemented to provide the 3D location/position of the target as with respect to subprocess 321. For example, at block 323, the optional subprocess 321 involves determining the position of the target anatomical feature using image data input from the endoscope camera, such as by implementing image processing using an artificial neural network or other process. When the target anatomical feature is identified in image data, the scope camera image may be annotated/overlaid with various visual features that identify the features in the video image for the user, such as bounding boxes, highlighting, text, arrows, masking, or the like. Identifier(s) may be displayed on the image to inform the user of the shape, form, and/or unique identifier value/number of the identified feature(s), as shown at block 326.

Once the position of the target anatomical feature has been assessed/determined, the process 300 may optionally proceed to block 325, where the determined target anatomical feature(s) may have a unique identifier associated therewith, such as an alphanumeric string, color, and/or other identifier.

The process 300 proceeds to subprocesses 330, which may involve tracking/localizing the target anatomical feature over an operative period while advancing a percutaneous-access instrument, such as a needle or the like, over/along an access path in the direction of the target anatomical feature. In some implementations, electromagnetic (EM) position-sensing technology is used to track/localize the target anatomical feature. For example, as described above, the target anatomical feature (e.g., papilla) may be contacted by the distal end portion of the scope at one or more positions/areas, wherein the local position and orientation of the target feature(s) (e.g., infundibular axis) may be determined based thereon with respect to position(s) of the scope. In some embodiments, a mapping of the target site (e.g., target calyx/papilla and associated infundibula) may be generated based on a plurality of recorded positions from EM sensor data.

The subprocess 330 may be implemented in various ways. For example, as shown as the subprocess 332, live direct instrument (e.g., scope) targeting/tracking may be implemented to provide operational tracking of the target anatomical feature. For example, throughout the relevant operative period, the distal end of the medical instrument may be maintained in contact with the target anatomical feature (block 334), such that position sensor data indicated by the medical instruments may provide a real-time accurate location of the target anatomical feature. Therefore, as shown at block 336, the live position of the medical instrument may be targeted to provide the desired percutaneous access path. However, with the distal end of the medical instrument in close proximity/contact with the target anatomical feature, real-time visualization of the target anatomical feature may not be possible or sufficiently clear due to the obstruction of the target anatomical feature by the feature itself in the field of view of the camera(s). That is, the camera associated with the local instruments may be sufficiently blocked or obscured by the mass of the target anatomical feature, thereby preventing the physician/user from having visual confirmation of penetration of the target anatomical feature by the percutaneous-access instrument (e.g., needle).

An alternative subprocess 331 is shown for tracking the target anatomical feature while still maintaining a clear visual of the target anatomical feature during approximation of the percutaneous-access instrument. The subprocess 331 involves localizing the target anatomical feature using a determined position offset/translation between the position of the scope and the position of the target anatomical feature and determining live/present position(s) of the target anatomical feature by applying the offset/translation to the present position of the scope.

At block 333, the subprocess 331 may involve recording the determined position of the target feature contact position associated with the contact with the target anatomical feature implemented in connection with the operation of block 320, described above. As an example, the user may provide input to notify the relevant control/medical system of the feature-contact position of the target anatomical feature by tagging/registering the position of the exposed face of the target anatomical feature (e.g., papilla face exposed within the target calyx) in some manner. Such tagging may be implemented through provision of user input in some manner or may be substantially automatic based on perceived tissue contact, or the like. The position data may be capturing in volatile and/or non-volatile data storage of certain control circuitry as shown and described herein.

After determining the location/position of the target anatomical feature, the scope may be retracted and/or parked in a manner such that it faces the target anatomical feature (e.g., papilla) to provide visualization thereof, as indicated at block 335. In some implementations, such parking may be performed with the aid of certain scope-guidance feature(s)/overlay(s) presented on or around/near a camera view interface window.

Rather than continuing to maintain the medical instrument (e.g., scope) in contact/proximity with the target anatomical feature to provide live operational tracking as with subprocess 332, the subprocess 331 may involve determining the position of the target anatomical feature based on a determined positional offset/translation between the position/orientation of the parked scope and the position/orientation of the target anatomical feature, as indicated at block 337. When parking the scope, the scope may be retracted a distance away (e.g., in the proximal direction) from the target anatomical feature to thereby allow the medical instrument to clearly capture the target anatomical feature in a field of view of the camera(s) associated therewith. For example, in some implementations, the physician/user may inform the system in some manner when the medical instrument has been parked a desired distance away from the target anatomical feature.

By way of clarification, it is noted that the subprocesses 331, 332 represent alternative implementations of the subprocess 330. That is, the process 300 may generally involve implementation of either the subprocess 332 or the subprocess 331, but not both.

In some cases, it may be assumed that as the scope remains inside of the target calyx, the papilla-to-scope offset/translation is generally preserved over time. In the absence of relative movement of the target anatomical feature with respect to the scope position sensor(s), the target position can be continuously updated based on determined current scope position. The position data (e.g., EM data) collected in connection with retraction/reorientation of the scope can be used to determine the offset/translation of the papilla location/orientation with respect to the scope. For example, in accordance with one use case, the retraction/positioning of the scope could be approximately 5 mm in front of the papilla and 2 mm to the left. Such position offset may be used to determine the position of the target as relative to a current position of the scope. The translation/offset information may further incorporate orientation information, which may be enabled in any suitable or desirable way. In the event of relative movement between the target anatomical feature and the scope, the determined offset/translation may become unreliability. In some implementations, relative movement compensation may be implemented to compensate for, and/or adjust, the offset/translation when the relative position/orientation between the scope and target anatomical feature changes.

The subprocess 331 may or may not include/involve the contacting 333 and retracting 335 steps, wherein the user physically contacts the target papilla location and retracts the scope to show the papilla in the field of view of the scope. For example, where image-based position-determination 321 is implemented in connection with block 320, it may not be necessary to physically contact the target anatomical feature to determine the position/location thereof. Rather, the position/location may be determined using target-identification mechanism(s) based on image data captured/generated by one or more cameras of the scope/instrument. For example, in some embodiments, the target is identified and tracked using multiple frames of image/vision and/or position (e.g., EM) data. In some implementations, by looking at the target anatomical feature (e.g., papilla) from two distinct positions and/or alignments, the target position can be estimated/determined with respect to three-dimensional space.

At block 339, the subprocess 331 involves targeting the tracked location of the target anatomical feature with the percutaneous-access instrument. For example, the centroid of an identified papilla shape or form in a real-time image of the treatment site may be used at the target position for a percutaneous-access instrument. At block 340, the process 300 involves puncturing the target anatomical feature, either without visual confirmation of the target anatomical feature with respect to the subprocess 332 or with visual confirmation in accordance with the subprocess 331, depending on the particular implementation of the process 300.

The various position sensors used in connection with embodiments of the present disclosure, such as for determining/recording the feature-contact position at block 333 or targeting the live instrument position at block 336, may be any type of position sensors. As an example, such sensor(s) may be electromagnetic (EM) sensors/probes. With respect to the scope, the position sensor may be attached or integrated with, proximal to, the tip thereof. Alternatively, the sensor(s) may comprise a coil connected to an electrical wire running the length of the scope, which is connected to external control circuitry configured to interpret electrical signals generated at the coil and passed down the wire. Examples of types of position sensor devices that may be implemented in connection with embodiments of the present disclosure include, but are not limited to, accelerometers, gyroscopes, magnetometers, fiber optic shape sensing (e.g., via Bragg gratings, Rayleigh scattering, interferometry, or related techniques), etc. Depending on the implementation, registration to a separate form of patient imagery, such as a CT scan, may or may not be necessary to provide a frame of reference for locating a urinary stone within the patient.

With respect to EM-type sensors, such as coils or other antennas, such sensor devices can be configured detect changes in EM fields as the EM sensor moves within the field (e.g., within the kidney). Therefore, certain embodiments are implemented using one or more EM generators configured to emit EM fields that are picked-up and/or affected by the EM sensor(s). The EM generator(s) may be modulated in any suitable or desirable way, such that when their emitted fields are captured/affected by the EM sensor(s) and are processed by appropriate control circuitry, signals from different EM generators are separable to provide additional dimensions/degrees-of-freedom of position information. EM generators may be modulated in time or in frequency, and may use orthogonal modulations so that each signal is fully separable from each other signal despite possibly overlapping in time. Further, separate EM generators may be oriented relative to each other in Cartesian space at non-zero, non-orthogonal angles so that changes in orientation of the EM sensor(s) will result in the EM sensor(s) receiving at least some signal from at least one of the EM generators at any instant in time.

With further reference to the recording of the feature-contact position at block 333 of FIG. 10, EM position data may be registered to an image of the patient captured with a different technique other than EM (or whatever mechanism is used to capture the alignment sensor's data), such as a CT scan, in order to establish a reference frame/space for the EM data. In addition to the scope, the percutaneous-access needle may include one or more position/alignment sensors, such as an EM sensor. Position/alignment data received from the needle EM sensor may be received and processed similarly to scope position data as describe above. It should be understood that the various processes described herein may be performed wholly or partially manually and/or wholly or partially using robotics.

The processes disclosed herein may be implemented in connection with procedures other than kidney stone removal procedures, such as gallbladder stone removal, lung (pulmonary/transthoracic) tumor biopsy, and others. Generally, any type of percutaneous procedure may be performed by using an endoscope configured to capture image data for feature identification and tracking using neural network processing in accordance with embodiments of the present disclosure. Additional examples include stomach operations, esophagus and lung operations, etc. Further, the objects to be removed do not necessarily need to be urinary stones, they may be any object, such as a foreign body or object created within the human body.

The process 300 can be implemented to localize the target anatomical feature based at least in part on the determination of scope offset/translation from the target anatomical feature, as may be implemented in connection with any of the embodiments disclosed herein. Electromagnetic sensor(s) incorporated in the distal end portion of the ureteroscope may have any suitable or desirable form and/or configuration, including one or more conductor coils, rings, cylinders, and/or the like, wherein local distortion in the broadcast electromagnetic field caused by such conductive element(s) can provide information relating to the position thereof.

Using an electromagnetic positioning system, including an electromagnetic field generator and one or more electromagnetic sensors/beacons, the present location of the papilla can be tracked to facilitate real-time targeting of the papilla by the percutaneous access instrument (e.g., needle). For example, the targeting position of the papilla may be updated in real-time based on electromagnetic sensor data, such as real-time electromagnetic sensor data relating to one or more sensors/beacons associated with the distal end of the endoscope. In some implementations, even in the absence of real-time visual confirmation and/or other image data associated with the scope and target anatomical feature, the position and/or orientation of the scope may be relied upon to determine the real-time tracking location for the target anatomical feature.

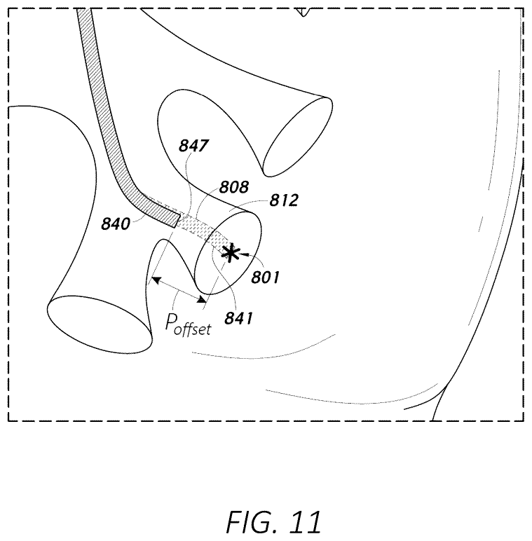

FIG. 11 shows a scope device 840 disposed within a target calyx 812 for target localization in accordance with one or more embodiments. Certain process(es) may be implemented to determine and/or maintain a known offset P_{offset} between a recorded/known papilla target position 801 and a present position of the distal end 847 of the endoscope 840 in a parked position. The position sensor(s)/beacon(s) of the scope 840 may be configured to provide sensor data indicating five or six degrees-of-freedom (DOF) with respect to the position of the scope 840. For example, coil or other type of sensor device(s) may have a cylinder-type shape, or any other shape allowing for three-dimensional position data as well as yaw and/or pitch data. In some embodiments, the position sensor(s)/beacon(s) do not provide roll information, such as in embodiments including five-DOF sensor(s). In some embodiments, multiple five-DOF sensors may be used/combined and disposed at a relative axial angle with respect to one another, wherein the combined data provided/generated based on such position sensor(s)/beacon(s) can define a plane that can be used to construct six DOF providing scope roll information.

In some implementations, a breath-hold may be executed for the patient during at least a portion of the scope offset determination/maintenance process(es), which may allow for such operations to be executed without the necessity of accounting for anatomical motion associated with the pulmonary cycle. For example, the patient may be subject to a breath-hold during at least the tagging and retracting portions of the process(es). In some implementations, it may not be necessary to update the determined translation P_{offset} in real time if it is assumed that any anatomical motion experienced after determination of the offset may affect the parked endoscope and the target anatomical feature (e.g., papilla) in a like manner, such that the transform/translation between the two positions can be assumed to be substantially constant irrespective of anatomical motion and/or other factors.

Determination of the relative transform P_{offset} between the parked scope end 847 and the previously-recorded papilla target position 801 can be determined using strictly electromagnetic position sensor data, or may be determined using image processing, as described herein. For example, calibration of the scope camera with respect to the electromagnetic field space may allow for visual determination of distance and/or position changes between the target position 801 and the parked scope position 847.

The path 808 of retraction between the position 841 of the scope and the position 840 of the scope may or may not be linear. In some implementations, such as with respect to lower-pole target calyces/papillas, the retraction path may be at least partially arc-like. Therefore, the translation P_{offset} may be determined with respect to more than just straight-line distance, and may incorporate scope orientation and/or other position-related parameters. Therefore, the translation P_{offset} may be considered a six-degrees-of-freedom translation/transform in some implementations. Such translation determination may account at least in part for cases in which the target calyx 812 and/or associated infundibulum may have a central axis/centroid that does not necessarily align with the retraction path taken by the scope 840. Therefore, translation with respect to six or more degrees of freedom may be desirable to produce a mapping translation/transform that sufficiently accurately represents the positional offset P_{offset} between the position 801 and the position 847.

In some implementations, certain image data may be collected and used for identifying target anatomical features. For example, systems, devices, and methods of the present disclosure may provide for identification of target anatomical features in real-time endoscope images, wherein identification of a target anatomical feature in an image may prompt certain responsive action. For example, control circuitry communicatively coupled to robotic endoscopy and/or percutaneous-access device(s) may be configured to track movements of a target feature and take action, such as articulating one or more portions of the endoscope (e.g., distal end portion), or adjusting target position data. For example, the control circuitry may be configured to cause the endoscope to articulate so as to center the target position/points at or near a center of the field of view of an interface and/or image field of the endoscope camera and/or to maintain a desired positional offset (e.g., P_{offset}) between the scope and the target anatomical feature.

By utilizing robotic-assisted percutaneous access, a physician may be able to perform operating target access and treatment. Furthermore, percutaneous access can be further assisted utilizing automated target identification and tracking in accordance with aspects of the present disclosure described in greater detail below, which may be relied upon for accurately maintaining the target position for percutaneous access guidance. Percutaneous access guided by scope-enabled target tracking in accordance with aspects of the present disclosure can be relatively less skill-intensive. In some implementations, a single operator or robotic system may carry out the process. Furthermore, the need for fluoroscopy can be obviated.

### Vision-Based Target Localization

Aspects of the present disclosure relate to vision-based processes that can be implemented to determine target feature position relative to endoscope position. In some implementations, multiple camera images and/or EM-based position data relating to the scope can be obtained at distinct positions/orientations with respect to the target anatomy, wherein the three-dimensional (3D) location of the anatomy with respect to the scope may be determined based on such data. Localization of a target anatomical feature (e.g., target papilla) may be achieved using any suitable image processing mechanisms/functionality. For example, control circuitry of the medical system may receive image data in real-time from the scope camera and run certain image processing processes/functionality thereon to identify the target anatomical feature(s). In some implementations, at least two separate images of the target anatomical feature(s) are processed in order to track the position thereof.

Generally, in percutaneous nephrolithotomy, the kidney is accessed through less vascularized areas called papillae in order to minimize blood loss and damage to the kidney. Access is achieved when the physician is able to guide a needle percutaneously towards a target placed near the target papilla inside the target calyx. In some implementations, the position of this percutaneous access target can be determined through contact-tagging the target anatomy and subsequently parking the endoscope at a position having a known transform/relationship relative to the tagged target (such as described with reference to FIGS. 6A and 8). Such implementations involve target determination/placement that depends on as few as two readings from the scope position sensors (e.g., EM sensors), without relying/utilizing information from visual data generated by scope camera(s).

The present disclosure provides various additional methods/mechanisms for determining the access target position using images captured by a camera disposed on (or coupled to) an endoscope. In some implementations, the present disclosure relates to processes for determining and utilizing target feature position in camera images, which can be extracted from visual data and fused with other position data (e.g., electromagnetic (EM) sensor data), to accurately track the feature (e.g., papilla) in three-dimensional space, even in the presence of physiological motion induced by e.g., respiration or needle insertion. In other words, the visual data can enhance or supplement the position information derived from the EM sensor data, which can improve the robustness and accuracy of target localization and/or tracking.

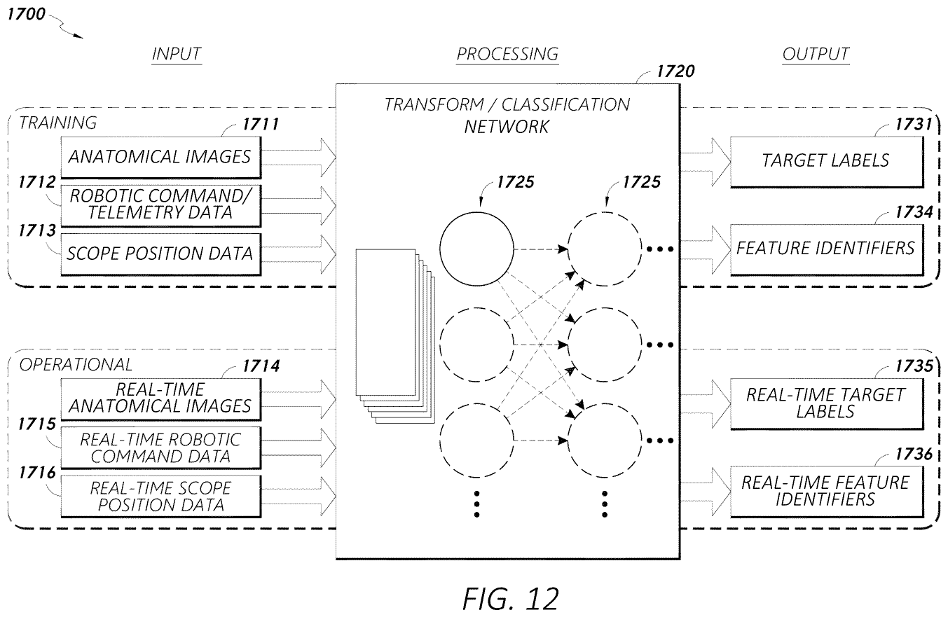

In some implementations, target identification in camera images may be achieved using a pretrained neural network configured to indicate the position of visible, relevant anatomical features (e.g., papillae) in images, and assign unique identifiers to each relevant identified feature. FIG. 12 illustrates a feature-identification framework 1700 for identifying one or more target anatomical features in endoscope camera images for dynamically updating target position data in accordance with one or more embodiments of the present disclosure. The feature-identification framework 1700 may be embodied in control circuitry, including one or more processors, data storage devices, connectivity features, substrates, passive and/or active hardware circuit devices, chips/dies, and/or the like. For example, the framework 1700 may be embodied in any of the control circuitry 251, 211 shown in FIGS. 3 and 4, respectively, and described in detail above. The feature identification framework 1700 may employ machine learning functionality to perform automatic target detection on, for example, ureteroscopic images of internal renal anatomy.

The framework 1700 may be configured to operate on image-type data structures, such as image data representing at least a portion of a treatment site associated with medical procedure(s). Such input data/data-structures may be operated on in some manner by a transform network 1720 associated with an image processing portion of the framework 1700. The transform network 1720 may comprise any suitable or desirable transform and/or classification architecture, such as any suitable or desirable artificial neural network architecture. Example suitable neural network architectures include convolutional neural networks (CNNs), recurrent neural networks (RNNs), and long short-term memory (LSTM) networks, among other examples.

The transform network 1720 is trained to produce target labels 1731 based on anatomical images 1711, for example, as input/output pairs. More specifically, the transform network 1720 is configured to adjust one or more parameters or weights associated therewith to correlate the known input and output image data. For example, the transform network 1720 can be trained using a labelled dataset and/or machine learning. In some implementations the transform network 1720 is trained based on robotic command/telemetry data 1712 and/or scope position data 1713 (e.g., EM data) associated with the anatomical images 1711. The framework 1700 can be configured to execute the learning/training in any suitable or desirable manner. For example, the framework 1700 can compare the outputs of the transform network 1720 with ground truth data associated with the inputs to determine a measure of “loss” between the outputs and the ground truth data. The framework 1700 can adjust the weights associated with the network 1720 to minimize the loss over one or more iterations (where each iteration can be performed using different input data).

The ground truth for the target labels 1731 may be generated at least in part by manually labeling anatomical features in the anatomical images 1711. For example, manual labels may be determined and/or applied by a relevant medical expert to label where, for example, papilla anatomy is among inter-calyx anatomical images. The known input/output pairs can indicate the parameters of the transform network 1720, which can be dynamically updatable in some embodiments. In some implementations, the transform network 1720 also may assign a unique feature identifier 1734 (such as “Papilla 1,” “Papilla 2,” “Midpole 1,” Midpole 2,” etc.) to each of the detected features associated with the target labels 1731. For example, the feature identifiers 1734 can also be learned from ground truth (such as manual annotations) associated with the anatomical images 1711.

The transform network 1720 can include a plurality of neurons (e.g., layers 1725 of neurons, as shown in FIG. 12) corresponding to overlapping regions of an input image that cover the visual area of the input image. The transform network 1720 can further operate to flatten the input image, or portion(s) thereof, in some manner. The transform network 1720 can be configured to capture spatial and/or temporal dependencies in the input images through the application of certain filters. Such filters can be executed in various convolution operations to achieve the desired output data. Such convolution operations can be used to extract features, such as edges, contours, and the like. The transform network 1720 can include any number of convolutional layers, wherein more layers may provide for identification of higher-level features. The transform network 1720 can further include one or more pooling layers, which may be configured to reduce the spatial size of convolved features, which can be useful for extracting features which are rotational and/or positional invariant, as with certain anatomical features. Once prepared through flattening, pooling, and/or other processes, the image data may be processed by a multi-level perceptron and/or a feed-forward neural network. Furthermore, backpropagation may be applied to each iteration of training. The framework may able to distinguish between dominating and certain low-level features in the input images and classify them using any suitable or desirable technique.

The input(s) to the transform network 1720 can comprise video or still images. In some embodiments, the input comprises video, wherein the transform network 1720 is configured to produce output that indicates how certain features of the video change over time (e.g., implementation of a time model). In some implementations, once the transform network 1720 has been trained, as a neural network model, operational input to the trained neural network model can comprise a plurality (e.g., two) of images, wherein the output comprises data indicating the locations of one or more targets across the images (e.g., over time). The input(s) can comprise images and/or data indicating differences between separate (e.g., consecutive) images. For example, the transform network 1720 can detect motion or spatial change between pixels, wherein a change in pixel position indicates motion between images and/or over time. The output can indicate the difference between images. In some embodiments, the transform network 1720 may be trained to estimate optical flow.

The framework 1700 can also include a feature-extraction network, such as a feature pyramid network, configured to extract one or more feature maps of an input image and/or engineer feature-detector contours. For example, multiple images can be processed with different levels of resolution to provide output variants. The framework 1700 can further comprise a region proposal network component configured to propose certain bounding boxes on an image that encapsulate targets indicated/identified on feature maps generated and/or provided by the feature extraction component. In some embodiments, a coarse-to-fine processing diagram can be executed to extract the target in an image. Proposed bounding boxes provided by the region proposal network can be used by one or more additional components. For example, a binary classification network component can be configured to classify bounding boxes based on whether the bounding boxes contain a target feature of interest. Further, a box regression network can be used to refine boundary boxes proposed by the region proposal network. In addition, a mask prediction network component can be used to calculate and/or represent the silhouette or shape/form of identified target feature(s).

During an inferencing phase, the framework 1700 can be used to generate or infer real-time target labels 1735 that indicate whether or not a real-time anatomical image 1714 includes a target anatomical feature. Images that are identified as containing one or more instances of a target anatomical feature can be passesed further downstream an image processing pipeline, for example, to identify the location and/or other aspects of the target anatomical feature. In some implementations, further processing may be performed to determine a three-dimensional location of the identified target anatomical feature and/or one or more portions thereof. As alternatives to traditional single-lens camera image data, the real-time anatomical images 1714 can comprise three-dimensional (3D) scanning data, depth camera data, stereo camera data, etc.

The framework 1700 can be further configured to generate or infer real-time target labels 1735 based on the real-time anatomical images 1714, real-time robotic command/telemetry data 1715, and/or real-time scope position data 1716 (e.g., EM position sensor data, shape sensing data, inertial measurement data, etc.) using the trained model of the transform network 1720. For example, during a medical procedure, real-time images of the treatment site, and/or real-time robotic/EM data, associated with an endoscope or other medical instrument disposed at a treatment site may be processed using the trained model of the transform network 1720 to generate the real-time target labels 1735 indicating the presence and/or position of one or more target anatomical features in the real-time images. The real-time target labels 1735 can comprise bounding boxes and/or masking visual features (such as binary masks) for overlaying/annotating on the real-time images.

As described, the inputs to the transform network 1720 can be images from the scope camera, robot telemetry data, electromagnetic sensor readings, and/or user inputs (such as for controlling movement of the scope). The outputs of the transform network 1720 can include real-time target labels 1735 indicating a location of each anatomical feature detected in the image and a real-time feature identifier 1736 for the detected feature. The outputs 1735, 1736 can be illustrated in a variety of forms. For example, the real-time target labels 1735 can include one or more bounding boxes having width and height dimensions that encompass/contain the detected anatomical feature(s), binary masks that classify pixels as being associated with the detected feature(s) or not associated with the detected feature(s), relaxed binary masks that assign confidence values to one or more pixels that indicating the network's confidence in classifying the pixel as being associated with the detected anatomical feature(s) or not associated with the detected anatomical feature(s). Relaxed masks can be particularly useful for detecting anatomical features with ambiguous boundaries that are difficult to delineate from background features. The real-time feature identifier 1736 for each anatomical feature can be a unique color for each bounding box or overlaid mask, a number, or a string.

In some implementations, real-time anatomical images 1714 may be processed by the transform network 1720 to identify the presence, position, and/or shape of a target papilla, which can be targeted with a percutaneous-access instrument. With respect to ureteroscopic applications, papillary anatomy presents similarly in appearance across subjects, and therefore papillary anatomy can be identifiable across a broad demographic.

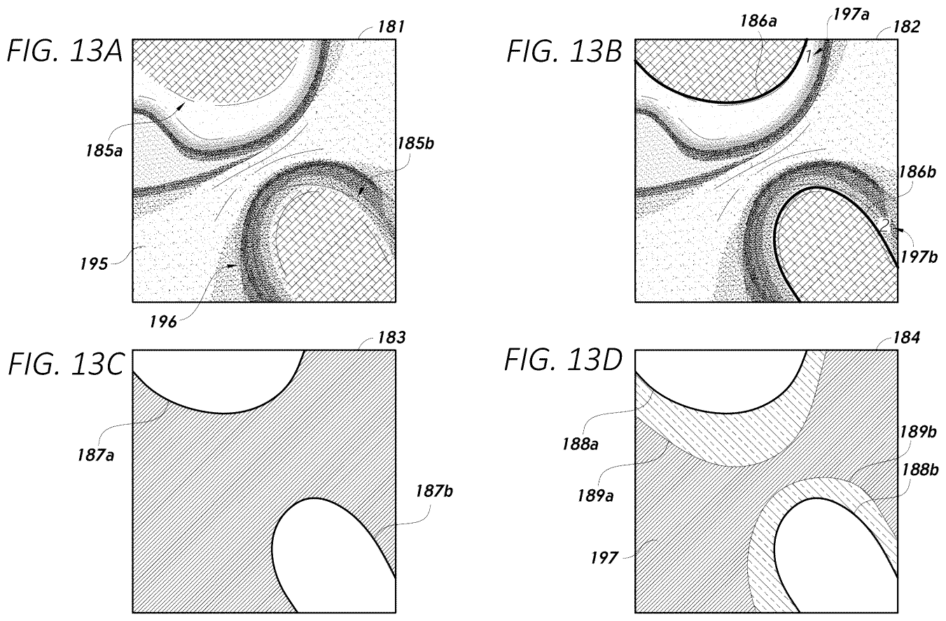

FIGS. 13A, 13B, 13C, and 13D show camera images of an anatomical site in accordance with one or more embodiments. As described above, control circuitry of robotic system in accordance with aspects of the present disclosure can be configured to implement certain image processing techniques to detect and identify target anatomical features in endoscope camera images. Such embodiments can involve generating graphical image data for display in connection with scope camera images, wherein the image data represents certain features that highlight the detected feature(s).

For reference, FIG. 13A shows an example endoscope camera image 181 displaying a window/field-of-view of an endoscope camera. The image 181 may be generated based on signals received from the endoscope camera when the distal end of the endoscope is disposed in a target anatomical chamber/site. The image 181 shows two identifiable anatomical features 185a, 185b (individually/collectively ‘185’), which may be anatomical features of interest (e.g., renal papilla viewable from a scope position within a calyx network) as potentially providing a desirable percutaneous access path therethrough. In the image 181, calyx infundibular tissue 195, and fornix tissue 196 around the papilla(s) 185.

FIG. 13B shows an image 182, which may be generated using output from a neural network or other framework as described herein, including anatomical feature identification markers 186a, 186b (individually/collectively ‘186’) overlaid on the endoscope video image 181 to highlight the identified anatomical features 185a, 185b, respectively. The marker(s) 186 can comprise lines or other features that follow the detected contours of the anatomical feature(s) 185. In addition to the contour lines/masks 186, identifier values/features 197 may be provided as annotations that ascribe a unique identification for each identified anatomical feature 185. Although shown as numbers, the identifiers 197 may comprise unique color markings, or a string.

FIG. 13C shows an image 183 including additional and/or alternative visual overlay(s) that may be generated to highlight for the user (e.g., surgeon) the presence, shape, size, and/or location of the identified anatomical features 185. In the example of FIG. 13C, the visual features have the form/configuration of a binary mask that highlights areas 187a, 187b (individually/collectively ‘187’) corresponding to pixels of the image 181 that represent the identified feature(s) 185. In some implementations, the binary mask comprises a relaxed binary mask, where each pixel has a corresponding confidence value indicating a determined confidence in classifying the pixel as a part of the anatomical feature(s) 185. Such a relaxed mask may be particularly useful for anatomical features whose boundaries are ambiguous and therefore difficult to precisely mark.

FIG. 13D shows an image 184 including additional and/or alternative visual overlay(s) that may be generated to highlight for the user (e.g., surgeon) the presence, shape, size, and/or location of the identified target features/papillae 185, forniceal tissue 196, and/or infundibular tissue 195. In the example of FIG. 13D, the visual features have the form/configuration of masking that highlights areas 188a, 188b (individually/collectively ‘188’) corresponding to pixels of the image 181 that represent the identified feature(s) 185. The visual features further include masking that highlights areas 189a, 189b (individually/collectively ‘189’) immediately around the target anatomical feature masks 188a, 188b, such as fornix tissue around the papilla. In some implementations, the mask areas 189 may represent tissue that is determined to be potentially part of the target feature(s) 185, but for which confidence of such identification is lower (e.g., between 10-50% confidence). The visual features of the image 183 may further include masking or other identification 197 for the tissue outside of the masked areas 188, 189, which may correspond to the calyx/infundibulum tissue, for example.

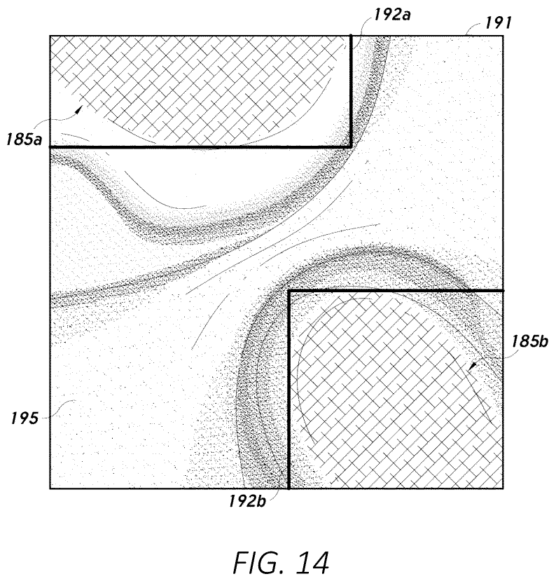

FIG. 14 shows another enhanced/annotated image 191 of a scope camera image of the example anatomical site associated with FIGS. 13A-13D. The image 191 includes bounding-box features 192a, 192b (individually/collectively ‘192’) in accordance with one or more embodiments. The bounding-box anatomical feature identification markers 192a, 192b (individually/collectively ‘192’) can be overlaid on the endoscope video image 181 (see FIG. 13A) to highlight the identified anatomical features 185a, 185b, respectively. The marker(s) 192 can comprise lines or other features that extend in straight lines in two axes/dimensions to contain the contours of all or a portion of the anatomical feature(s) 185. The width and height of the boxes 192 can advantageously be large enough to contain at least the visible portions of the anatomical feature(s).

With the assistance of visual enhancement features as shown in any of FIGS. 13A-13D and 14, a physician can have knowledge of the location, shape, and/or unique identifier of each target anatomy (e.g., papilla) while driving an endoscope (e.g., ureteroscope) during, for example, a tag-park localization workflow as described herein. The visual features of FIGS. 13A-13D and 14 can be generated based on visual data from the scope's camera.

### Scope Positioning/Parking Guidance

As described above, in certain nephrolithotomy procedures, before obtaining percutaneous access through the kidney with a needle, the physician may first place a target inside the target calyx towards which the needle is guided to create a percutaneous tract/pathway. Such processes can be facilitated by incorporating visual data from the camera on the ureteroscope in accordance with aspects of the present disclosure to provide guidance for identifying the target anatomy and parking the endoscope in a desirable position for viewing and tracking the target anatomy.

Some systems and examples disclosed herein provide the ability to determine a physician's/user's intent regarding selection of a desired target anatomical feature among feature(s) represented in scope camera images. Such intent/selection can be utilized to assist the physician in properly tagging the correct anatomical feature (e.g., papilla) and/or parking the endoscope at a desirable position relative to the selected target. The present disclosure provides various mechanisms for allowing the physician/user to interact with a robotic system to show intent. For example, in some implementations, the user can signal anatomical feature selection intent by maintaining an identified anatomical feature (e.g., feature identified using camera image processing as described above) centered within the endoscope camera image for a predetermined/fixed period of time. In some embodiments, user selection intent may be determined by displaying a timer visual features, such as a progress bar, a countdown timer, or the like, when a potential target feature is positioned over a center of the scope camera image, wherein elapsing of the timer associated with the timer feature with the target feature centered on the camera image throughout the timer period triggers determination of user intent as selecting the target feature. Once the fixed period of time has elapsed with the anatomical target feature (e.g., papilla) centered successfully, the scope camera display can be modified such that only the bounding box, mask, or other anatomy-identifying feature(s) implemented relating to the selected target are displayed.

As an alternative mechanism for determining the user selection/intent, the robotic system may include a touchscreen display, wherein the user may touch the display in the area of an identified anatomical feature to select the feature as the percutaneous access target. For example, such touch input may be within a bounding box, outline, or mask of the desired feature (e.g., papilla). In some implementations, intent may be signaled by the user by centering the desired feature in the scope camera image and pressing a button or other input when the feature is over the center of the image. The center of a camera image may be highlighted for the user by a reticle or other visual feature to assist the user in selecting the desired target feature.

Once the desired anatomical feature has been selected as the target feature or otherwise identified/determined by the system control circuitry, the control circuitry can provide visual guidance to the user using camera image overlays or other output to assist the user in parking the scope at a desirable position to include the target feature at a suitable position within the field of view of the camera and/or to maintain a suitable transform between the position of the scope and the target for localization purposes.

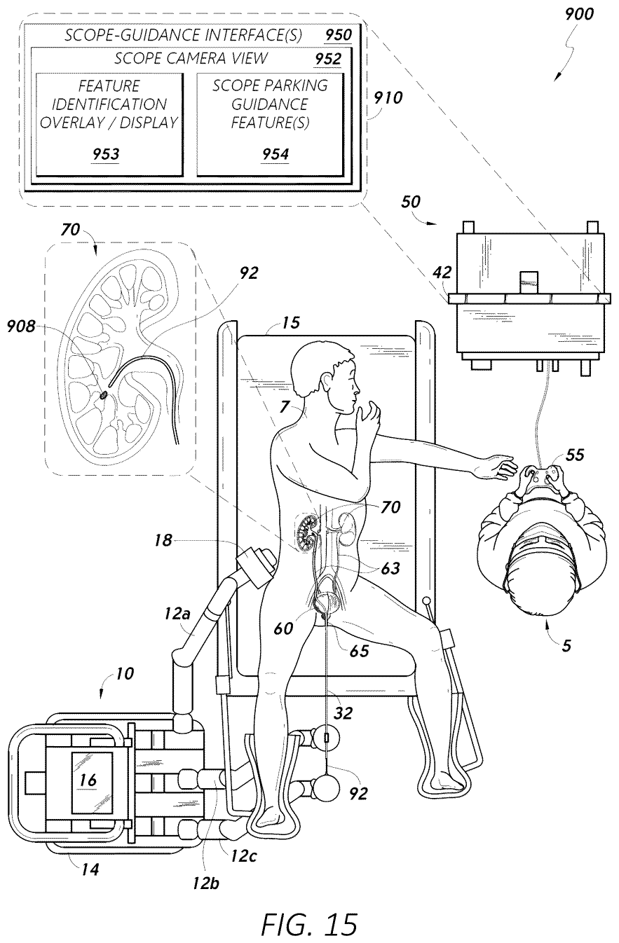

FIG. 15 illustrates a robotic medical system 900 arranged to facilitate navigation of a scope within a patient in accordance with one or more embodiments. For example, the physician 5 can connect an endoscope 92 to a robotic arm 12c of a robotic system 10 and/or position the scope 92 at least partially within a medical instrument (e.g., catheter/sheath) and/or the patient 7. The scope 92 can be connected to the robotic arm 12c at any time, such as before the procedure or during the procedure (e.g., after positioning the robotic system 10). The physician 5 can then interact with a control system 50, such as with the control device(s) 55, to navigate the scope 92 within the patient 7. For example, the physician 5 can provide input via the control device(s) 55 to control the robotic arm 12c to navigate the scope 92 through the urethra 65, the bladder 60, the ureter 63, and up to the kidney 70.

As shown, the control system 50 can present a screen/interface 910 including a scope camera view window 952. As disclosed in detail herein, the scope camera window 952 can have displayed thereon certain feature-identification overlays 953 and/or scope parking guidance overlays/features 954. The scope camera view 952 provides intraoperative real-time camera video 952 captured by the scope 92, which can be supplemented by the identification and/or guidance overlay(s)/feature(s) to assist the physician 5 in controlling the scope 92. Various examples of the feature-identification features 953 and parking/navigation guidance features 954 are provided in the figures of the present disclosure. The features 953, 954 can advantageously ease the physician's scope driving experience while minimizing any user-introduced error.

The physician 5 can navigate the scope 92 to locate, for example, a kidney stone, target anatomical feature(s), and/or the like. In some embodiment, the control system 50 can be configured to implement certain localization technique(s) to determine a position and/or an orientation of the scope 92, which can be viewed by the physician 5 through the display(s) 42 to also assist in controlling the scope 92. Further, in some embodiments, other types of information can be presented through the display(s) 42 to assist the physician 5 in controlling the scope 92, such as x-ray images of the internal anatomy of the patient 7. The physician 5 can use the controls 55 to drive the scope 92 to find/identify the kidney stone 908 or other artifact targeted for removal/treatment. The physician 5 may further drive the scope to localize the target papilla and to occupy a desired parking position. Such scope driving can be guided at least in part by the scope parking guidance feature(s) 954 of the scope-guidance interface(s) 950.

According to the present disclosure, certain visual indicators/cues can be provided to the user to guide the user's navigation of the robotic endoscope for scope parking, such as by guiding the user to navigate the endoscope in such a manner as to position the distal end of the scope such that the target anatomical feature (e.g., papilla) occupies a minimum or maximum fraction of a demarcated area of the camera image/display, such as a reticle area. Parking guidance as disclosed herein can help ensure that the user/physician has not parked the endoscope (e.g., ureteroscope) too close or too far from the target anatomical feature. Furthermore, with knowledge of the target anatomical feature's location and shape, as determined using the image processing techniques disclosed above, a reticle (e.g., crosshair), or other visual pointer, can be overlaid on the camera image to guide the user in positioning the center of the camera field of view over a central area/point on the target anatomical feature.

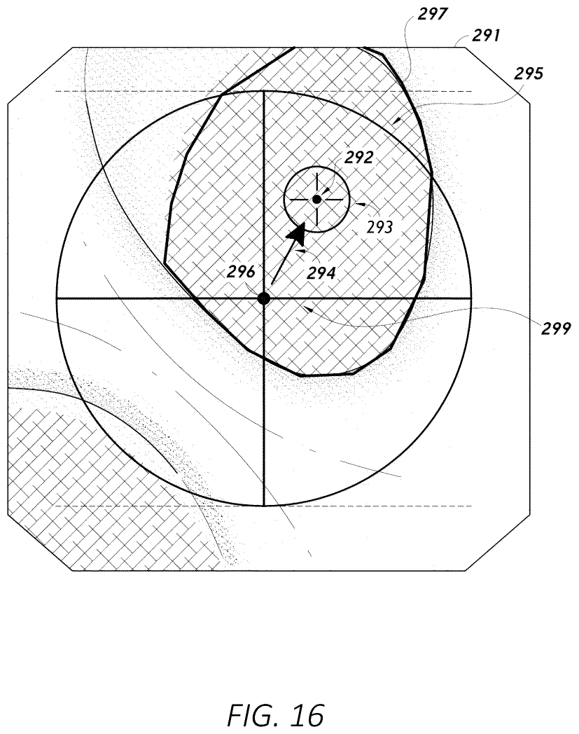

FIG. 16 illustrates a scope camera view/window 291 including endoscope positioning guidance features in accordance with one or more embodiments. As illustrated, scope parking guidance features may be overlaid on a scope camera video image, and may include one or more reticles for directing positioning of the scope camera, and hence the scope, in the area of the center of the target anatomical feature 295 (e.g., papillar). For example, a first reticle 299 may be superimposed on the image that points to the center 296 of the camera view frame. Although shown as a crosshair reticle, it should be understood that the feature 299 may have any shape, form, or configuration that visually indicates the center 296 of the image. The anatomical feature 295 may have an outline 297, mask, or similar identification feature as described herein.

The guidance features, additionally or alternatively, may include a second reticle 293 that is positioned automatically at a center of the target anatomical feature 295 identified using a neural network framework as described herein, or any other identification mechanism. For example, the reticle, or other type of visual indicator that indicates/points-to the center of the feature 295, can be positioned at a center 292 of mass of the feature 295, or a center of the feature 295 along an axis of interest (e.g., longitudinal axis of the feature 295). The determination of the center of the feature 295 can be performed automatically in connection with the anatomical feature identification processes disclosed herein. With the reticle 293 presented in the center of the target anatomical feature 295 and the other reticle 299 placed in the center of the scope camera view 291, the user can aim the scope to align the reticle (e.g., crosshair) 293 on the target anatomical feature 295 with the reticle 299 in the center of the scope camera view 291, which can advantageously result in an optimal parking position and/or orientation for the scope. An arrow 294 or other directional feature may provide guidance to the user directing the user in the appropriate direction to maneuver the scope towards the center 292 of the feature 295.

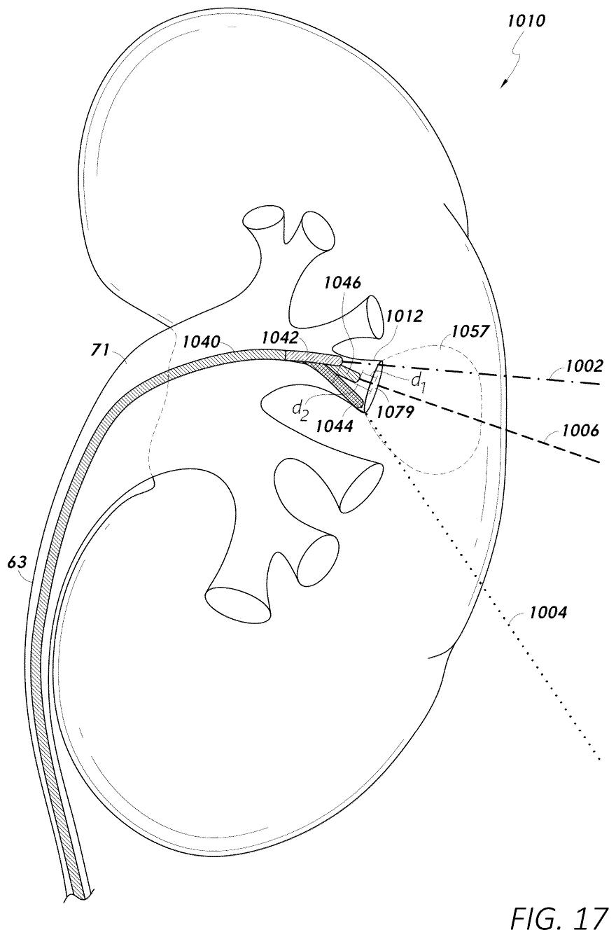

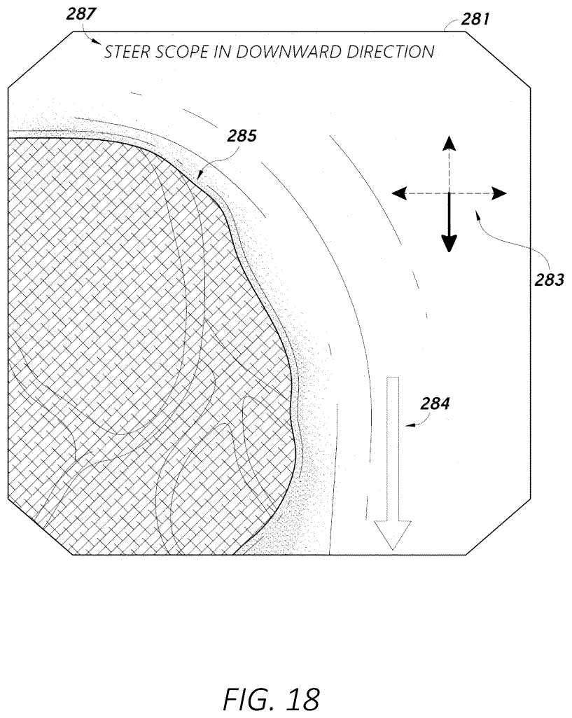

Guided centering of the feature 295 in the scope camera view 291 can ensure that the ureteroscope is not parked at an oblique angle from the target papilla 295 and is aligned coaxially with the papilla. Such alignment can be beneficial, as misalignment with the percutaneous access target can result in an undesirable access path for the percutaneous instrument. For example, FIG. 17 shows a ureteroscope 1040 disposed within the ureter 63, renal pelvis 71, and/or calyces (e.g., major and/or minor calyces) of a kidney 1010. An operator/physician may drive the scope 1040 to the calyx 1012 and use an electromagnetic beacon associated with a distal end/tip of the scope 1040 as a target to which a percutaneous access instrument (e.g., needle) may be directed. Generally, when the scope 1040 is parked, one or more points/sensors at or near the distal tip/end of the scope 1040 may be used as the target for percutaneous access. The illustration of FIG. 18 shows three different possible example parking positions (1042, 1044, 1046) of the distal end of the scope 1040, and further shows a respective coaxial trajectory (1002, 1004, 1006) associated with each of the scope parking positions. Such trajectories may be determined based on the derived position and/or orientation/alignment information relating to each of the scope parking positions and represent possible paths along which percutaneous renal access can be guided/achieved.

The parking position 1046 corresponds to the distal end of the scope 1040 being generally aligned with a center axis 1006 of the target calyx 1012 and/or associated infundibulum, with the scope positioned an optimal distance d₁ from the papilla 1079. The parking position 1046 corresponds to a position where the distal end of the scope 1040 is parked a distance d₂ that is undesirably far away from the papilla 1079 and/or having a trajectory 1002 that is misaligned with the central axis 1006 of the target calyx 1012. The parking position 1044 that is misaligned with the axis of the target calyx 1012, papilla 1079, and/or associated infundibulum, wherein the trajectory 1004 is deflected far from the papilla center 1079. The features 293, 299 of FIG. 16, when implemented, can reduce the risk of the user navigating to a scope parking position that does not provide an optimal percutaneous access target path, such as a ureteroscope at or near a target calyx within a kidney of a patient.

Embodiments of the present disclosure provide additional examples of user feedback/output the user/physician with guidance about how to best position and orient the endoscope. After the physician indicates which papilla is the desired one, there are various implementations within the scope of the present disclosure for providing assistance while the user is driving the ureteroscope to tag the target anatomy and/or park the scope after tagging or otherwise determining the target position. For example, haptic feedback on the user input controller can be provided, such as through a pendant or other control input mechanism, wherein the control system automatically impedes input engagement (e.g., joystick movement) in a direction moving away from the target anatomy (e.g., movement that causes a target papilla to move off-screen or away from the center of the image). In some implementations, such as when the target anatomy moves away from the center of the camera image (e.g., moves off-screen), “virtual walls” may block any controller/pendant input that would result in the target anatomy moving further away from the center of the camera image. In other words, may prevent the scope from moving away from the target anatomy. Such robotic control modification can simulate for the user the experience of the endoscope hitting a wall, such that it cannot move further in that direction.

FIG. 18 illustrates a scope camera view/window 281 including anatomical feature location guidance features in accordance with one or more embodiments. The directional features superimposed on the scope camera view 281 can be used to direct a user to control an endoscope to a desirable position vis-à-vis a target anatomical feature, whether for tagging/recording the position of the feature or for parking the scope at a desirable position and orientation relative to the target anatomical feature. In some implementations, the control system/circuitry is configured to generate and provide visual cues on the scope camera image notifying the user that the scope is positioned too far away from the target anatomy, and/or is oriented at an undesirable orientation, as reflected by the scope camera field of view not capturing the target anatomy in a central position. To direct/redirect the user back towards the target anatomical feature (e.g., papilla), an arrow 284 or other similar pointing feature may be implemented that points off-screen in the approximate direction of the target anatomy relative to the camera field of view to inform the user of where the target anatomy is located relative to the current camera view, and therefore the current scope position. In some implementations, a textual message 287 may be presented that indicates the user inputs or directions for manipulating the scope to the desired position. Additionally or alternatively, a directional coordinate frame 283 may be displayed with the appropriate direction indicated to guide the user's scope control. The colors of one or more of the directional guidance features 283, 284, 287 can also indicate how far the target anatomy is off-screen in the indicated direction. In the context of nephrolithotomy, the various features of FIG. 18 can help the physician reliably tag the target papilla rather than the fornix or infundibulum. The scope camera view 281 may include one or more identified anatomical features 285 other than the target feature/area.

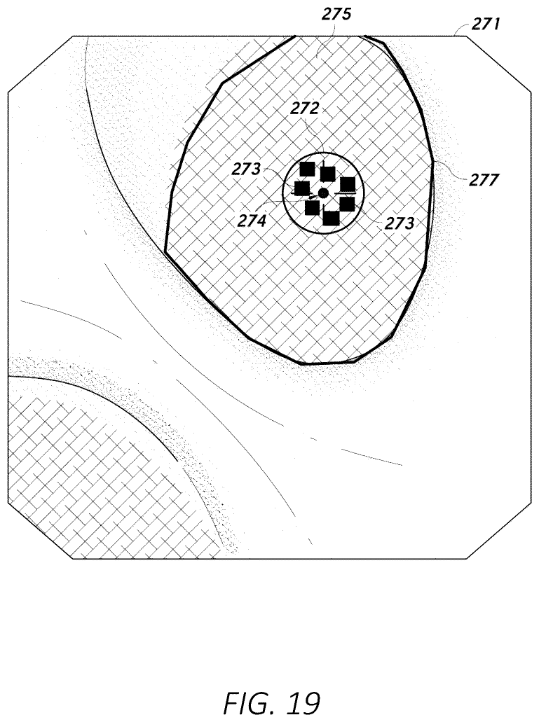

As described in detail above, an alternative to implementing a neural network framework to automatically locate target anatomy in camera images can involve allowing the physician to actively designate where he/she would like to tag the target anatomy for localization/targeting. For example, before parking the endoscope, the user/physician may touch on the screen at a point on the target anatomy where he/she would ideally like to make contact with the scope and/or where the user would like to set the percutaneous access target. FIG. 19 illustrate a scope camera view/window 271 including anatomical feature tracking point 274 corresponding to a position/point where a user has touched or otherwise identified as a point/location wherein he/she would like to tag the target anatomy 275 in accordance with one or more embodiments. The tracking point 274 may be selected by the user, wherein the target feature 275 is highlighted/identified by an identifier feature 277, which can be an outline, bounding box, or any other feature contemplated herein. Once the target point 274 has been recorded, a neural network framework and/or other control circuitry can then be used to identify and track generic features 273 in a target area 272, rather than the target anatomical feature/papilla 275 itself, around the selected point 274 from one image to the next. That is, rather than focusing on the target anatomy feature 275 as a whole, the system can ensure that the general location chosen by the user/physician is approximately centered or does not drift off-screen by tracking more-easily identified and/or tracked features in proximity to the selected point 274. For example, features 273 in the scope camera view 271 that would be most successfully tracked may include areas of pixels that have easily discernible texture and/or are in high contrast relative to surrounding pixels. By tracking the features 273 rather than, or in addition to, tracking the selected point 274 or the feature 275 as a whole, the efficiency and accuracy of the target tracking can be improved.

### Vision-Based Target Triangulation

Described above are various techniques for tagging a target anatomical feature for percutaneous access targeting and endoscope positioning/parking after tagging for improved targeting. In some implementations, the present disclosure provides alternatives for locating and tracking target anatomical features using triangulation based on multiple camera views/images taken from different positions/orientations relative to the target anatomy. Such multiple-image processes can be implemented to determine three-dimensional (3D) positioning of the target anatomy.

According to aspects of the present disclosure, multiple camera images and EM-based position data relating to an endoscope can be obtained at distinct positions/orientations with respect to the target anatomy (e.g., papilla), wherein the 3D location of the target anatomy with respect to the scope may be determined based on such data. Localization of the target anatomical feature (e.g., target papilla) can be achieved using any suitable image processing mechanisms/functionality. For example, control circuitry of the medical system may receive image data from the scope camera and run certain image processing processes/functionality thereon to identify the target anatomical feature(s). In some implementations, at least two separate images of the target anatomical feature(s) are processed in order to track the position thereof. When implementing target triangulation, the user need not drive the endoscope to contact the target anatomy and then park.

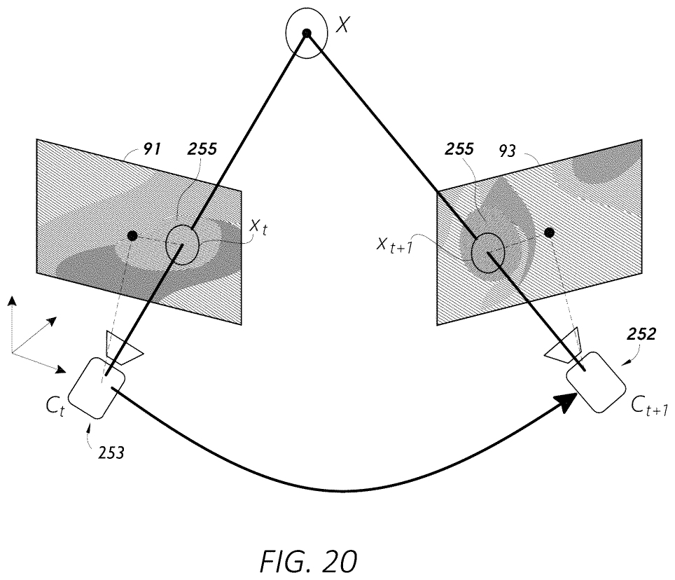

FIG. 20 illustrates a three-dimensional position estimation framework in accordance with one or more embodiments. As shown in FIG. 20, an image 91 including a target anatomical feature 225 is captured from a first perspective/position 253 of the scope camera, and a second image 93 is captured by the scope camera either before or after capture of the first image 91. More specifically, the second image 93 is captured from a different perspective/position 252 of the scope camera. In some implementations, structure-from-motion techniques can be implemented to determine the three-dimensional position of the target anatomical feature 255 based at least in part on the images 91, 93.

Three-dimensional (3D) position estimation for the purpose of target anatomical feature localization in accordance with aspects of the present disclosure may be implemented in accordance with any suitable or desirable technique or mechanism. For example, in some embodiments, distance between an endoscope camera and a target anatomical feature may be estimated based on the representative size of the anatomical feature 255 in an image. In some embodiments, information relating to angle of movement of a scope and/or anatomical feature may be used to determine 3D position. For example, electromagnetic sensors/beacons in an electromagnetic field/space can provide such angle of movement information. By combining electromagnetic sensor data with image data, mappings between the distance from the target anatomical feature and size of the target anatomical feature in a resulting image captured after the movement of such distance can be used to estimate depth/distance of features in subsequent images. In some embodiments, when contacting the target anatomical feature (e.g., a papilla) and retracting the scope away from such feature to park the scope in a position to provide a desirable field-of-view, the distance traveled may be registered using, for example, electromagnetic sensor data. Furthermore, subsequent images can provide information relating to how large the anatomical feature appears in such images, and therefore the relationship/mapping between feature size and distance can be determined and used for future position determination. In some implementations, machine learning may be utilized to classify images and determine position information based on the size of features in such images.

In some embodiments, certain sensor(s) associated with medical instruments (e.g., scopes) can be utilized to obtain the 3D location of the target 255. For example, structured-lighting sensor(s) and/or time-of-flight sensor(s) can be used in determination of 3D positioning. In accordance with some embodiments, a geometric translation approach may be implemented to detect the 3D position of a target anatomical feature. For example, as with certain other embodiments of the present disclosure, images 91, 93 may be captured that are associated with separate timestamps. In connection with such images, rotational translation information with respect to the camera can be determined based on sensor information from any suitable or desirable sensor or device and used to triangulate and/or determine the positions of such images in 3D space, thereby providing information indicating 3D location of target anatomical feature 255 in the 3D space. The rotational translation information can be based on robotic actuator movement and/or position sensor information, such as from an electromagnetic beacon device associated with the camera and/or scope and indicating a position of the camera in the electromagnetic field space.

Given the intrinsic and extrinsic parameters (principle points, focal length and distortion factors, relative motion) of the camera, the 3D location of the target anatomical feature 255 can be calculated based at least in part on the tracked target two-dimensional (2D) locations on the images 91, 93. For intrinsic parameters, the camera principle point and focal length can be accounted for. Additional data that can be taken into account include radial and tangential distortion factors. Based on the sensor readings (e.g., robotic- and/or EM-based), extrinsic parameters can also be obtained, including rotation R and translation T of the scope between the locations where the two images were taken. For convenience, K can be denoted as a matrix that contains the intrinsic parameters and H denoted as a 4-by-4 matrix that contains the extrinsic rotation and translation between the camera position of the first image (Cₜ) and the camera position of the second image (Cₜ₊₁₎.

For Cₜ, the 3D-to-2D projection relationship can be expressed as xₜ=KX, where X is the 3D coordinate w.r.t. Cₜ and xₜ is the 2D coordinate (detected centroid of a target) on image t. Here, K is a three-by-4 matrix that can be expressed as:

$$K = [ \begin{matrix}{K}_{( 1 )} \\ {K}_{( 2 )} \\ {K}_{( 3 )}\end{matrix} ] ,$$

with K₍ₙ₎ being the n-th row in K.

Similarly, for Cₜ₊₁, xₜ₊₁=K′X, where:

$${K}^{′} = KH = [ \begin{matrix}{K}_{( 1 )}^{′} \\ {K}_{( 2 )}^{′} \\ {K}_{( 3 )}^{′}\end{matrix} ] .$$

As xₜ and KX are parallel vectors, xₜ×KX=0, and similarly, xₜ₊₁×K′X=0. Here, ‘x’ is the cross-product operator. Hence:

$${x}_{t} \times KX = 0 \text{=>} det [ \begin{matrix}i & j & k \\ {u}_{t} & {v}_{t} & 1 \\ {K}_{( 1 )} X & {K}_{( 2 )} X & {K}_{( 3 )} X\end{matrix} ] = 0 ,$$

The above can produce: i(vₜK₍₃₎X−K₍₂₎X)−j(uₜK₍₃₎X−K₍₁₎X)+k(uₜK₍₂₎X−vₜK₍₁₎X)=0, where uₜ and vₜ are the 2D coordinates of xₜ. Hence:

$${v}_{t} {K}_{( 3 )} X - {K}_{( 2 )} X = 0 \\ {u}_{t} {K}_{( 3 )} X - {K}_{( 1 )} X = 0 \\ {u}_{t} {K}_{( 2 )} X - {v}_{t} {K}_{( 1 )} X = 0$$

Here, only the first two equations are needed, as the third equation is a linear combination of the first two. Similarly, for Cₜ₊₁, the following two equations can be obtained:

$${v}_{t + 1} {K}_{( 3 )}^{′} X - {K}_{( 2 )}^{′} X = 0 \\ {u}_{t + 1} {K}_{( 3 )}^{′} X - {K}_{( 1 )}^{′} X = 0$$

After stacking the equations of Cₜ and Cₜ₊₁, the following can be produced:

AX=0,
where A is 4-by-4 matrix:

$$A = [ \begin{matrix}{v}_{t} {K}_{( 3 )} - {K}_{( 2 )} \\ {u}_{t} {K}_{( 3 )} - {K}_{( 1 )} \\ {v}_{t + 1} {K}_{( 3 )}^{′} - {K}_{( 2 )}^{′} \\ {u}_{t + 1} {K}_{( 3 )}^{′} - {K}_{( 1 )}^{′}\end{matrix} ] .$$

As the elements in A are known (detected 2D coordinates, intrinsic and extrinsic parameters), X can be calculated by performing singular value decomposition (SVD) on A:

A=UΣV^{T},
and the last column of V is the solution of X.

Therefore, in view of the foregoing disclosure, the various inventive concepts disclosed herein can be utilized to perform automatic target localization, including target detection, target tracking, and/or three-dimensional position estimation. In some embodiments, aspects of the present disclosure advantageously allow for target anatomical feature tracking without requiring physical contact with the target anatomical feature, which can facilitate improved ergonomics of the usage of the ureteroscope.

In some implementations, structured light and/or other non-contact optical sensing mechanisms, such as optical coherence tomography, or other interferometry technology, may be used to determine depth/offset information. Such techniques can advantageously provide 3D papilla/calyx location information. However, structured-like devices can be relatively large and thus increase the profile of the scope configured therewith. In some embodiments, the scope comprises a time-of-flight camera configured to emit light and receive reflections thereof, where the time between the emission and reception of light may be used to determine distances of objects and/or surfaces within the kidney.

By using multiple camera images to triangulate the position of the target anatomical feature, tagging the target anatomy through contact and/or parking a particular offset position/orientation may not be necessary. For example, in some use cases, a physician drives a ureteroscope to various calyces to select an optimal calyx/papilla as the target calyx/papilla. The manner in which the physician chooses the target papilla can be any of the various methods described above. In some implementations, a neural network framework can be implemented to locate all papillae in the scope camera image, wherein the physician can center the desired one or simply click on it on a screen or using any suitable input mechanism. In some implementations, the physician can select the target by clicking on a specific feature in the image to track. In order to then determine the target papilla's position without contact-tagging it, some implementations of aspects of the present disclosure involve maneuvering the endoscope tip to a plurality of different poses while visual data and position data of the scope is collected. It may be beneficial for the target papilla to be in view and able to be detected during this process.

At each pose, the ureteroscope maintains its position without being commanded for at least the duration of one respiration cycle. However, this constraint can be relaxed in some implementations through the use of a respiration motion model, which advantageously provides a relationship between the motion of the ureteroscope and the motion of a papilla throughout a respiration cycle. The respiration motion model can be constructed based on historical case data and/or by instructing the physician to hold the ureteroscope against the target for at least one respiration cycle. The position data of the ureteroscope can provide insight about how the papilla moves. With the respiration motion model, the motion of the ureteroscope, which can be generally known using position data, can provide an approximation of the papilla's motion. As a result, instead of collecting data across multiple respiration cycles and only using data points from a few specific phases, the scope can be constantly moving and collecting usable data because the papilla motion at any phase in the respiration cycle can be related to some reference phase of interest.

In order to accomplish target triangulation, the scope may be manipulated in multiple poses, which can be done in various ways. In some implementations, scope pose maneuvering may be performed in accordance with user interface guidance. In some other implementations, scope pose maneuvering may be performed at least partly autonomously.

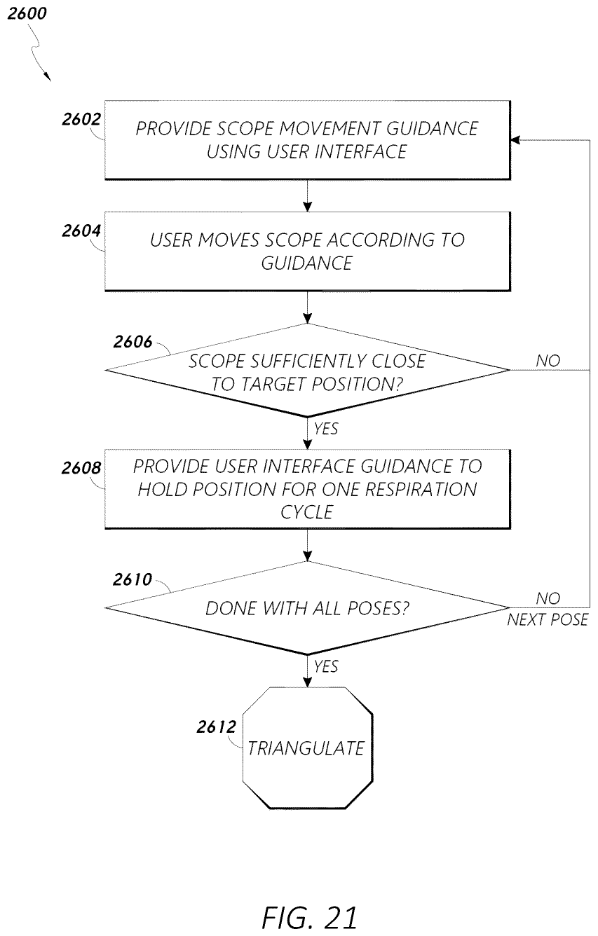

FIG. 21 is a flow diagram illustrating a process 2600 for triangulating a target anatomical feature using user interface guidance in accordance with one or more embodiments. At block 2602, the process 2600 involves providing scope movement guidance using one or more user interfaces. For example, such user interface guidance may include any directional or other instructional guidance as disclosed in connection with any example embodiment herein. At block 2604, the process 2600 involves the user controlling movement of the scope according to the guidance, such as through engagement with user robotic scope navigation controls. Such operation(s) can involve receiving user control commands using a user input controller/device and executing the commands in some manner to control the movement of the scope.

At block 2606, the process 2600 involves determining whether the scope is presently sufficiently close to the current pose being instructed. That is, in each iteration of the loop of the process 2600, a target position be instructed for a separate current pose of a plurality of poses for camera image triangulation. If the scope is not yet sufficiently close to the target pose position, the process 2600 may proceed back to block 2602, where additional scope movement guidance may be provided to the user to promote closer approximation of the scope to the target position. When the scope, through interface guidance as described herein, assumes a position close enough to the target position to satisfy triangulation requirements, the process 2600 may proceed to block 2608, where further user interface guidance may be provided to the user instructing the user to hold the present position of the scope for at least one respiration cycle to provide sufficient information to track the movement of the target anatomy over the different phases of the respiration cycle. Whether the present position and/or orientation of the scope is sufficiently close to the goal pose can be determined by system control circuitry automatically based on the three-dimensional (3D) position difference/error and/or the rotational difference/error associated with the present pose of the scope compared to the goal pose being smaller than a preset threshold. This cycle can be repeated for the number of required poses, and after data is collected at every pose, calculating the 3D position of the target feature via triangulation can be completed.

At block 2610, the process 2600 involves determining whether all necessary or desirable poses have been implemented to provide sufficient camera image data for target triangulation as described herein. If not, the process 2600 may loop back to block 2602, where the next pose may be instructed to the user. When all poses are complete, the image data generated in connection with scope camera image capture at each of the assumed poses may be utilized, at block 2612, to triangulate the three-dimensional position of the target anatomy without the need to tag the target anatomy through endoscope contact therewith.

In connection with processes disclosed herein for pose maneuvering guidance, as in the process 2600, the system can give cues/guidance to the physician indicating how he/she should maneuver the scope to reach the predetermined/known sequence of poses. These cues/guidance can come in the form of numbers indicating how far away in millimeters (or degrees for rotation), such as along the x, y, and/or z axes, the ureteroscope's end effector is from the desired pose or in the form of more generic directions, such as ‘up,’ ‘down,’ ‘left,’ ‘right,’ ‘forward,’ ‘backward,’ etc. These cues/guidance can be overlaid on whatever screen the physician uses for navigation.

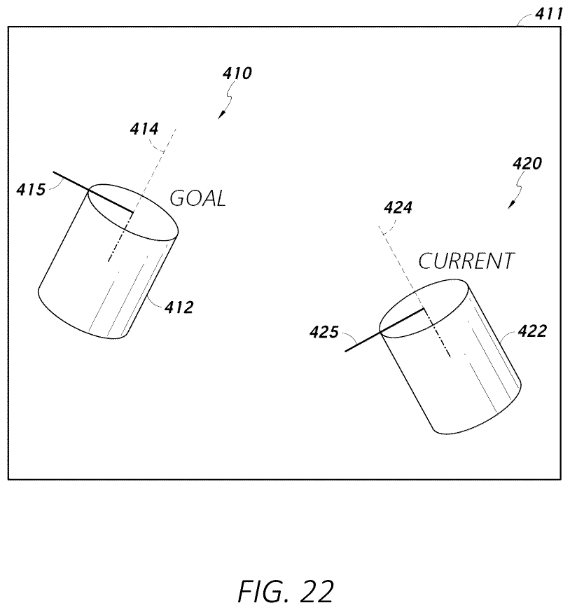

Other examples of scope maneuvering guidance can include providing a third-person point of view (POV) representing the ureteroscope's current end-effector pose and the desired end-effector pose. For example, FIG. 22 illustrates a graphical interface 411 representing third-person-perspective endoscope positioning guidance in accordance with one or more embodiments.

The example interface 411 shown in FIG. 22 provides unique information to the physician that the first-person POV provided by the camera on the endoscope generally cannot show. From a first-person POV, the physician may not be able to determine what the current pose of the endoscope looks like, and therefore it can be difficult for the physician to determine how the ureteroscope should be maneuvered to reach its goal pose 410. The interface 411 advantageously shows a representation 420 of a current pose 420 of the endoscope (e.g., ureteroscope) in a representation that indicates a position and/or orientation of the scope. For example, the vertical and/or horizontal position of the scope representation 420 within the interface 411 may indicate a position of the scope. Accordingly, moving/advancing the scope forward may move the icon 420 in an upward direction (or other direction depending on the particular implementation) with respect to the orientation of the interface 411 in FIG. 22; other directional movement may cause movement of the icon 420 in a corresponding relative manner in the interface 411. The cylinder icons 422, 412 represent the distal ends of an endoscope in the current 420 and goal 410 poses, respectively. The visual images in the interface 411 can direct the user to maneuver the scope 420 to more closely overlap and/or align with the icon representation of the goal pose 410, wherein movement of either or both of the icons 420, 410 as the user maneuvers the scope can provide immediate visual feedback to the user to guide the user towards the goal pose 410. The icons 410, 420 may have certain axis and/or coordinate frame icons 414, 415, 424, 425 associated therewith, which can provide further assistance to the user in aligning the orientation of the scope 420 to the goal pose orientation 410.

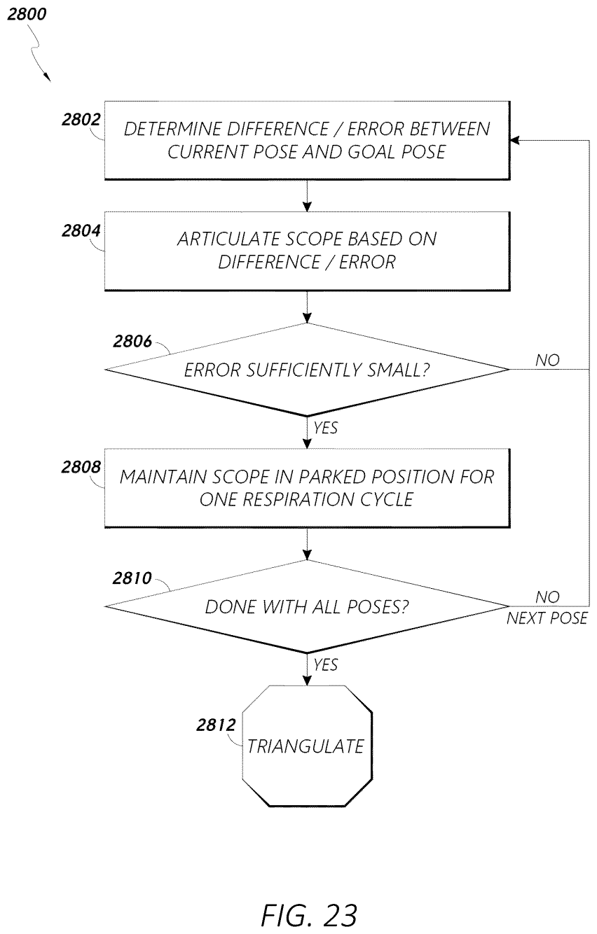

FIG. 23 is a flow diagram illustrating a process 2800 for autonomous triangulation of a target anatomical feature in accordance with one or more embodiments. At block 2802, the process 2800 involves determining an amount of difference/error between a current pose/position/orientation of an endoscope and a target/goal pose/position/orientation of the endoscope, wherein the goal pose represents one pose of a plurality of poses necessary or desirable for capturing camera image triangulation data as described in detail herein. The determined/calculated error can be used as input to a closed-loop controller (such as a proportional-integral-derivative (PID) controller), which can determine/calculate the appropriate control inputs such that the error is minimized.

At block 2804, the process 2800 involves automatically articulating or otherwise maneuvering the scope based on the determined difference/error between the current pose and the desired pose. For example, certain control circuitry may be utilized to generate robotic commands, such as advancement/retraction commands and/or articulation commands to cause movement of the endoscope in a manner as to reduce the error/difference between the present pose and the desired pose. The iterative automatic control associated with block 2804 may involve articulating/maneuvering the endoscope a relatively small amount according to the calculated control inputs, wherein a new error is determined/calculated when the scope reaches its new pose. This loop can continue until the distal end of the scope converges sufficiently close to its desired pose. Other aspects of the process 2800 can be similar to the aspects of the process 2600 described above.

At block 2806, the process 2800 involves determining whether the scope position error has been reduced sufficiently by the automatic control of block 2804. If not, the process 2800 may proceed back to blocks 2802 and 2804 to determine the amount of present difference/error and/or automatically robotically control/maneuver the endoscope to minimize the difference/error based thereon. Whether the present position and/or orientation of the scope is sufficiently close to the goal pose can be determined by system control circuitry automatically based on the three-dimensional (3D) position difference/error and/or and the rotational difference/error associated with the present pose of the scope compared to the goal pose being smaller than a preset threshold.

When the scope position difference/error becomes sufficiently small, the process 2800 may proceed to block 2808, where the automatic robotic control of the endoscope may be paused for a predetermined period to maintain the scope in the present parked position for at least one respiration cycle to allow for image generation relating to the target anatomy over the various phases of the respiration cycle. Once all poses have been commanded/performed automatically in connection with blocks 2802-2810, the process 2800 may proceed to block 2812, where triangulation of the target anatomical feature may be implemented as described in detail herein. Unlike the various user-interface-guided scope maneuvering solutions disclosed herein, the autonomous scope maneuvering process 2800 for target feature triangulation may not require intervention by the physician.

After the visual and position data are collected at each pose, data processing can be performed to extract the images and position data at the times when the papilla is at a specific position, as triangulation can be challenging with respect to a moving target. Respiration gating can be used to extract the data corresponding to a specific phase in each respiration cycle. In some implementations, it may be assumed that the target anatomy (e.g., papilla) has the same position for a specific phase across subsequent respiration cycles.

Whatever features are extracted from the visual data can be used as the features that are triangulated. Such features can correspond to, for example, the center of the target anatomy (e.g., papilla), a grid of points spread across the target anatomy, points along the contour(s) of the target anatomy, physician-chosen points, or other point(s). The precise location of these features in multiple images can be determined by control circuitry configured to determine/calculate how pixels move between different frames, such as dense or sparse optical flow determination. Through triangulation, the three-dimensional positions of these features can be determined.

Triangulation can be implemented in various ways according to aspects of the present disclosure, each of which potentially providing different amounts and/or type of information about the target anatomy. For example, in some implementations, a single feature of the target anatomy can be triangulated across one phase in a plurality of respiration cycles. This can provide the three-dimensional (3D) location of the single feature during one phase of the respiration cycle. In some implementations, a single feature can be triangulated across multiple phases in multiple respiration cycles. Such implementations can involve extracting more visual and position data and performing triangulation for each desired phase in the respiration cycle. Generating such information about the feature's 3D location across multiple phases can provide an estimated trajectory for that feature. In some implementations, multiple features from the target anatomy can be triangulated for a single phase in multiple respiration cycles, which can provide 3D positions for multiple points on the target anatomy. The orientation of the target anatomy (e.g., papilla) can then be determined/estimated by fitting a plane among those points and calculating the plane's normal vector. In some implementations, multiple features from the target anatomy can be triangulated for multiple phases in multiple respiration cycles, which can provide 3D positions of said features, as well as anatomical feature orientation, along with a trajectory of how the feature positions and, e.g., papilla orientation change over the course of a respiration cycle.

### Determination of Pose Positions/Orientations and Number of Poses for Triangulation

Examples of the present disclosure provide solutions for triangulating target anatomical features using image data associated with a plurality of poses directed towards the target features. Robotic systems disclosed herein can comprise control circuitry configured to determine how many poses the scope is to be maneuvered to, and further what poses the scope should be maneuvered to for the purpose of triangulation.

In order to determine the number of poses to which the ureteroscope should be maneuvered, an uncertainty metric may be implemented relating to target feature imaging. For example, various sources of uncertainty may be accounted for with respect to the uncertainty metric, such as uncertainty associated with the position of the ureteroscope, the neural network or other image processing framework that is implemented to identify/detect the target anatomy (e.g., papilla), the mechanism used for matching features across multiple images, and/or the optimization calculation done for triangulation. In cases where there is relatively high uncertainty among onc or more of these sources, more data may be collected to reduce the triangulation uncertainty. Uncertainty can be quantified and used to filter out low-quality data, wherein the uncertainty metric can be determined based on the collected visual data to quantify how well features are being tracked between multiple images. In some examples, the uncertainty metric is based on measured uncertainty in the estimated 3D positions from triangulation.

Measuring the uncertainty in how well features can be tracked between two images can come in multiple forms. The first option is using an image similarity measurement where the assumption is that if images are too dissimilar, then features from one image most likely are not well-tracked in the other image. If a pair of images has high similarity or contains a set of features that have been well-matched, then both images along with their corresponding position data can be used in triangulation. If not, then more poses should be obtained until some minimum number of data points have been collected. In addition, another measurement can come from the confidence of the neural network in its classifications. Within a pair of images, if one or both contain papilla bounding boxes or masks with low confidence values, then there is a high likelihood that the features being triangulated may not be on the papilla or that the features between both images do not match. The estimated 3D position would then be completely inaccurate and give the physician a false sense of where the target papilla is located.

After the visual data is filtered, uncertainty may remain related to the position data acquired at each pose that the endoscope was commanded to. The present disclosure provides various mechanisms for minimizing such uncertainty to determine the ideal number of poses and obtain an accurate triangulated three-dimensional (3D) position of the target. In some implementations, a fixed number of poses may be used. Generally, for triangulation, a minimum number of two poses are required, while a higher number of poses (e.g., five or six) may provide better triangulation accuracy. With the data collected at each of the implemented poses, a resampling technique, such as random sample consensus (RANSAC) sampling, can be utilized to discount/discard outlier data and obtain a more precise triangulated 3D position. In some implementations, a dynamic number of poses can be used. For example, the endoscope may first be navigated to three different poses. Triangulation can then be executed using data from different combination (e.g., every combination) of those three poses. In some implementations, if there is a cluster of position estimates with a desired density (or uncertainty), then no additional poses may be used, as further data may be deemed unnecessary. If not, the endoscope can be maneuvered to one or more additional poses, wherein triangulation can be executed again with every additional combination of data that includes the most recently added pose(s). The larger number of position estimates can then be observed to determine if there is a cluster with a desired density (or uncertainty).

With respect to the determination of what positions and orientations should be associated with the various poses used to triangulate the target anatomical feature(s). In some implementations, at each stage, control circuitry is utilized to determine the next best view of the endoscope to obtain the best result for triangulation. Criteria that may serve as a basis for pose determination can include data indicating the size of the anatomical target (e.g., papilla), the location of the anatomical target in the camera image. The size of the target in the image can provide a useful relative measurement of how far away the scope is from the target anatomy, while the location of the target anatomy in the camera image can provide an indication of a relative measurement of how the scope is articulated. For example, if a papilla is first seen in the upper left corner of the image and then later in the lower right corner of the image, it can be estimated that the ureteroscope has articulated up and to the left. If the kinematics of the ureteroscope is known, or if there is an available robust motion model for the scope, a number of poses can be randomly sampled within the scope's reachable space. In some implementations, the best next pose can be determined as the pose that provides the greatest variance in the ureteroscope's position and orientation with the target anatomy kept in the field of view of the camera and therefore detectable by the system's circuitry.

In addition, or as an alternative, to using two-dimensional image detection criteria, such as size and location of the target anatomy (e.g., papilla) in the image view, on which to base pose determination, three-dimensional (3D) criteria may be used to make such determinations. For example, in a case where the feature(s) of interest is/are triangulated with a dynamic number of poses, a state estimation process/mechanism can be used to track the estimated 3D positions of these features and uncertainty associated with such estimated positions. In accordance with the present disclosure, such a state estimator can comprise a system including control circuitry configured to generate an estimate of an internal state of a system and model uncertainty based on measured data and/or noise inherent in the system. Some examples of parametric state estimation processes/mechanisms that may be implemented include Kalman filters, extended Kalman filters, non-parametric filters such as particle filters and histogram filters, and the like. The system may be configured to determine/select the next pose to implement by minimizing the values in the associated covariance matrix. For example, the system may be configured to calculate the eigenvalues and eigenvectors of each covariance matrix associated with the 3D triangulated position of a feature. Such eigenvalues can represent the magnitude of uncertainty, while the eigenvectors represent the direction of uncertainty. After the eigenvalues and eigenvectors of each feature's covariance matrix have been determined, the direction of highest uncertainty can be found, and the next pose can be determined as one that is close to orthogonal to said direction.

In some implementations, a next endoscope pose may be determined by re-projecting the current estimate of the target anatomical feature's 3D positions into the image space of every, or many, sampled pose from the endoscope's reachable space. By treating the re-projection as a nonlinear observation function, the covariance matrix of the state estimator can be propagated appropriately, such as in an extended Kalman filter. The best pose to use can be determined as the one that minimizes the magnitude of the updated covariance matrix.

In addition, even without strictly quantifying the uncertainty or covariance of the 3D triangulated features, the present disclosure provides various processes/mechanisms for determining the next endoscope pose for target triangulation. For example, in a case where a grid of two-dimensional (2D) target points spread across the scope camera image that the physician would like to triangulate, as the endoscope navigates to different poses, all of the 2D points may not be visible across each image. Therefore, next pose determination can involve maximizing the visibility of the 2D features/points across the images. An equivalent criteria could also be formulated in three-dimensional (3D) space. For example, where initial estimates of the 3D positions of a subset of the 2D points of interest have been determined, because not all of the 2D points are visible across multiple images, only a subset of 3D positions may be calculated. Based on this subset, sparse areas (or areas with missing points) in 3D space can be detected. The control circuitry may determine the best next pose as the one that maximizes visibility of these sparse areas so that the features in those spaces can be triangulated as well. Other constraints/parameters may also be considered when selecting the scope's next pose, such as minimizing travel distance or guaranteeing visibility of a minimum fraction of target feature points.

As an alternative to designating a specific pose to which the physician is guided to navigate, in some implementations, the user/physician may be instructed, using graphical interface guidance, to move the endoscope so that the size and location of the target papilla changes while still remaining within the image frame such that the target is still able to be detected by the image processing circuitry (e.g., neural network). Then, when the size and location of the target in the image frame have sufficiently changed, as determined by a static or dynamic threshold, the physician can be instructed to stop moving the scope as data is collected for a respiration cycle.

According to the triangulation workflow presented herein, the anatomical target can be localized in a number of ways. For example, before beginning the workflow, a two-dimensional (2D) target can be designated, and the corresponding three-dimensional (3D) position of said 2D target can then be calculated after collecting data at each pose. For example, the physician can touch a location on the display screen showing the camera image to indicate the feature of which he/she would like the corresponding 3D position, or the designated 2D target could simply be the center of a bounding box, mask, or other identification feature of the interface identifying the desired target anatomy (e.g., papilla). In some implementations, a target may not be designated in the image space before the triangulation workflow. In such implementation, if only one feature has been triangulated, then the target can be placed at the halfway point between the triangulated position and the current scope position. If multiple features on the target anatomy have been triangulated and the orientation of target anatomy is calculated, the target can be placed a fixed number of millimeters in front of the centroid of the triangulated 3D points, the peak of the triangulated 3D points (point that is furthest along the normal vector of target anatomy), or the triangulated 3D point with the least uncertainty.

After the target is localized, updating the target position as the kidney and ureteroscope are displaced due to physiological motion provides can provide certain benefits. For example, the accuracy of the target position may not be greatly affected due to the scope (e.g., ureteroscope) being displaced with respect to the anatomy (e.g., kidney). In addition, because the positional update of the target involves updating the position of the target anatomy (e.g., papilla), the target can be placed with respect to the target anatomy as opposed to the endoscope. With respect to nephrolithotomy applications, even in instances where the ureteroscope is kicked out of the target calyx, the position of the target can remain fixed with respect to the target papilla instead of following the ureteroscope and also being displaced out of the target calyx. Also, when the needle is inserted percutaneously, visual data can indicate how the papilla has moved, and the target can be updated reliably.

### Percutaneous Sheath Visual Overlay

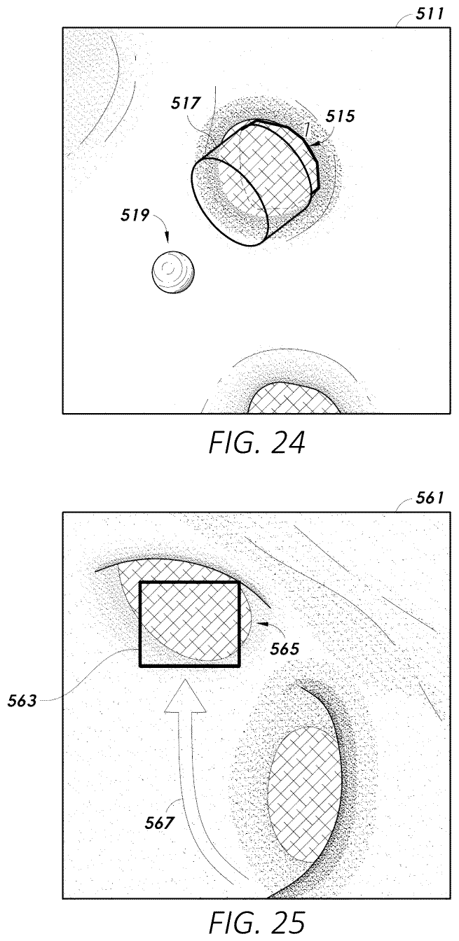

For nephrolithotomy applications, after the percutaneous access target localized as described above, and the three-dimensional position and orientation of the target papilla are approximately known, providing the user a visual indication of where the to-be-inserted percutaneous access sheath is to be positioned relative to the target anatomy can be helpful and is contemplated in the scope of the present disclosure. FIG. 24 illustrates a scope camera view/window 511 including instrumentation tracking features in accordance with one or more embodiments, such features including an overlay icon 517 representing a percutaneous access sheath superimposed on the target papilla 515, as well as an icon 519 indicating a location of the percutaneous access target as determined through tagging, triangulation, or other mechanism disclosed herein.

In embodiments in which the dimensions of the percutaneous access sheath and the orientation of the target papilla 515 are known, the sheath icon 517 can be overlaid in a pose that resembles the visual of the sheath in the proper orientation and position as it is anticipated in view of the position of the target 519. The sheath overlay 517 can give the user/physician a visual understanding of whether or not the target papilla 515 is a good or ideal candidate based on the representation 517 of the sheath's pose and ability to reach the necessary kidney stones from such position/orientation. The overlays 517, 519 can be color-coded to indicate how good of a candidate the target papilla 515 is. For example, the candidacy quality of an anatomical target can be based at least in part on how close the pose of the overlaid sheath 517 is to coaxial with the ureteroscope, as axial alignment can be helpful for a successful needle insertion. The target icon 519 can appear as a sphere or other shape/form.

Certain examples of the present disclosure provide endoscope camera views that include visual overlay features indicating a path of the scope's most recent positions. Such features can be useful to account for events during a surgical procedure where a large and/or unexpected physiological motion occurs, such as a ureteroscope being kicked out of the target calyx. In such cases, it can be helpful to provide the user interface guidance for returning to the previous scope position. FIG. 25 illustrates a scope camera view/window 561 including endoscope positioning guidance features in accordance with one or more embodiments.

The scope camera view 561 includes a path feature 567 overlaid on the scope camera view 561, wherein the path feature 567 is represented as an arrow. The scope camera view 561 can include a target identification feature 563, such as a bounding box or similar feature, on and/or around the target anatomy 565. When the endoscope is unexpectedly displaced from a parked position, the most recent data from the scope's position sensor(s) can be re-projected onto the image in addition to the path 567 to aid the user/physician in following the path back to the previous position. The path icon 567 can have the form of a curve or a simple pointing arrow. And once the endoscope is back in position, if there are multiple papillae within view, the unique identifiers provided by the aforementioned neural network image processing can be displayed for each papilla, informing the physician which one is the same desired target papilla as before.

### Localization Target Updating

After the percutaneous access target has been localized/placed at its initial location in accordance with any embodiment disclosed herein, further updating of the target may advantageously be implemented to account for motion of the target anatomy on account of respiration, both before insertion of a percutaneous needle, as well as after needle insertion. Prior to needle insertion, target updating can be implemented by triangulating features across multiple phases. For example, the location of the target anatomy (e.g., papilla), and therefore the target, can be updated in the triangulation workflow by triangulating the tracked features on the target anatomy across multiple phases of the respiration cycle to create an estimated trajectory. The target position can then be updated according to the estimated trajectory.

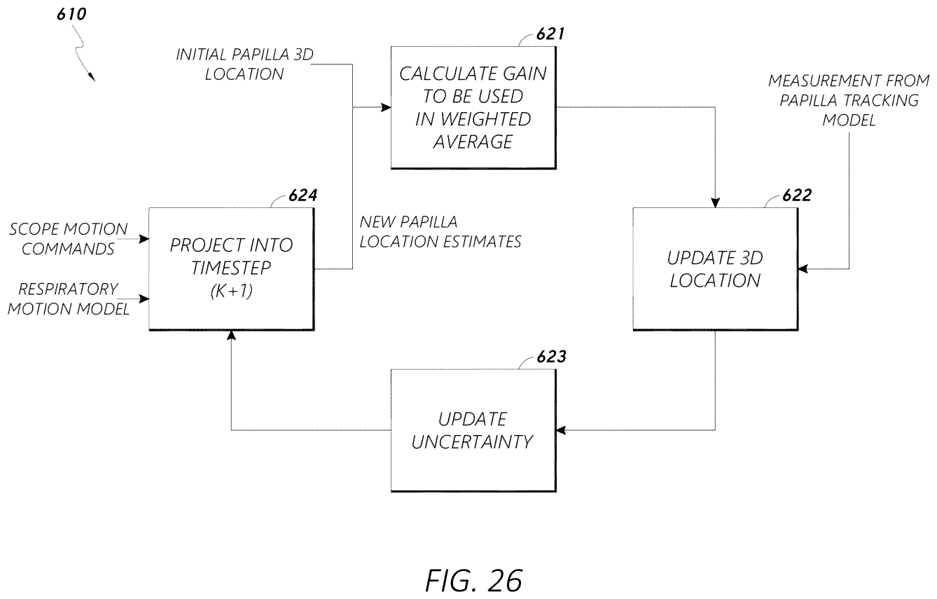

In some implementations, target updating can be achieved with a more accurate estimation of the papilla's location using state estimation methods, such as a Kalman filter. For example, the trajectory generated by target triangulation calculations can act as a state transition function, where the target anatomy's location in the next timestep can be approximately predicted based on the phase of the respiration cycle. FIG. 26 is a block diagram of an anatomical feature tracking framework 610 in accordance with one or more embodiments. The framework 610 includes blocks representing functional elements of a state estimator with an available respiratory motion model.

By detecting the papilla using image processing means as described in detail herein, the target anatomy (e.g., papilla) can be directly observed and used to correct target location prediction. The fusion of prediction and observation can generate more accurate and certain estimates of the target location. The framework 610 advantageously provides a mechanism for updating the target location that does not rely on triangulating features across multiple phases of a respiration cycle by using a respiration motion model to compensate for respiration effects. Because the motion model provides the relationship between ureteroscope motion and the motion of the target anatomy, the tagged/determined percutaneous access target initial location can be updated according to the estimated anatomical motion. In FIG. 26, kidney anatomy is referenced as an example, though the framework 610 may be utilized in other applications. In implementation in which visual data is available, a state estimator method can be used to more accurately estimate the papilla's location. In this case, the respiration motion model can act as the state transition function.

In the framework 610, an initial target anatomical feature 3D location may be updated using a calculated gain in a weighted average calculation as shown at block 621. Measurement from an anatomical feature tracking model may be provided to at block 622 to supplement and update the determined 3D location, and uncertainty metric(s) may be determined, at block 623, in connection with such location. Further scope motion commands and/or respiratory motion model parameters may be utilized to provide new anatomical feature patient estimates in an iterative fashion, as shown at block 624. Such new estimates may be combined with previous location to adjust the location in a weighted manner.

Updating of the target can account for motion of the target anatomy on account of respiration during percutaneous needle insertion. Control circuitry of the relevant robotic system may be configured to implement a state estimator according to the following relationships, where equation (1) corresponds to location prediction; equation (2) corresponds to location updating; xₜ represents state variables of the estimated 3D position of the target anatomy; u_{cmd} represents input from the user/physician through robotic (e.g., pendant) controller input to move the scope; u_{needle} represents disturbance introduced by needle insertion; u_{phys} represents papilla motion caused by physiological motion, such as respiration; zₚᵤₚᵢₗₗₐ represents a measurement of target anatomical feature (e.g., papilla) location through a tracking model; and fₘₑᵤₛ represents a measurement function to calculate measured target anatomical feature 3D location.

$${x}_{t} = {x}_{t - 1} + f ( {u}_{c m d} ) + g ( {u}_{n e e d l e} ) + h ( {u}_{phys} ) \qquad ( 1 )$$

$${x}_{t} = {x}_{t} + H ( {x}_{t} - {f}_{m e a s} ( {z}_{p a p i l l a} ) ) \qquad ( 2 )$$

In periods where, u_{needle} may be omitted. As the physician inserts the needle, the motion of the target papilla can change because the disturbance introduced by the needle affects its motion more greatly than respiration. Using a similar method as the state estimation method mentioned previously, the state transition function can provide a model that describes the relationship of the papilla's motion in the image space given knowledge about the needle's motion (such as needle's position and velocity). The observation corresponds to where the papilla is seen in the scope camera image. The predicted state of the papilla can be fused with the observation to provide a more accurate and certain state. And based on this fused state of the papilla's location, the target can be adjusted accordingly. An example formulation of this state estimator can be seen in equation (1) above.

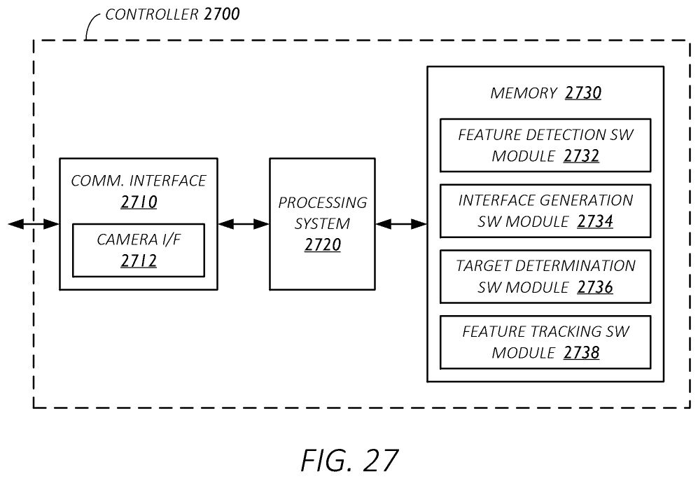

FIG. 27 shows a block diagram of an example controller 2700 for a robotic system, according to some implementations. In some implementations, the controller 2700 may be one example of any of the control circuitry 251 and/or 211 of FIGS. 2 and 3, respectively. More specifically, the controller 2700 is configured to track a target anatomical feature based on images captured by a camera associated with an instrument (such as an endoscope) coupled to a robotic manipulator.

The controller 2700 includes a communication interface 2710, a processing system 2720, and a memory 2730. The communication interface 2710 is configured to communicate with one or more components of the robotic system. More specifically, the communication interface 2710 includes a camera interface (I/F) 2712 for communicating with the camera associated with instrument. In some implementations, the camera I/F 2712 may receive an image depicting a field-of-view (FOV) of the camera.

The memory 2730 may include a non-transitory computer-readable medium (including one or more nonvolatile memory elements, such as EPROM, EEPROM, Flash memory, or a hard drive, among other examples) that may store the following software (SW) modules: a feature detection SW module 2732 to detect an anatomical feature in the image; an interface generation SW module 2734 to display a graphical interface that includes the image and a visual overlay identifying the anatomical feature in the image; a target determination SW module 2736 to determine whether the anatomical feature is a target anatomical feature based at least in part on a position of the visual overlay relative to the image; and a feature tracking SW module 2738 to track the anatomical feature based at least in part on determining that the anatomical feature is a target anatomical feature.

The processing system 2720 may include any suitable one or more processors capable of executing scripts or instructions of one or more software programs stored in the controller 2700 (such as in the memory 2730). For example, the processing system 2720 may execute the feature detection SW module 2732 to detect an anatomical feature in the image. The processing system 2720 also may execute the interface generation SW module 2734 to display a graphical interface that includes the image and a visual overlay identifying the anatomical feature in the image. The processing system 2720 may execute the target determination SW module 2736 to determine whether the anatomical feature is a target anatomical feature based at least in part on a position of the visual overlay relative to the image. The processing system 2720 may further execute the feature tracking SW module 2738 to track the anatomical feature based at least in part on determining that the anatomical feature is a target anatomical feature.

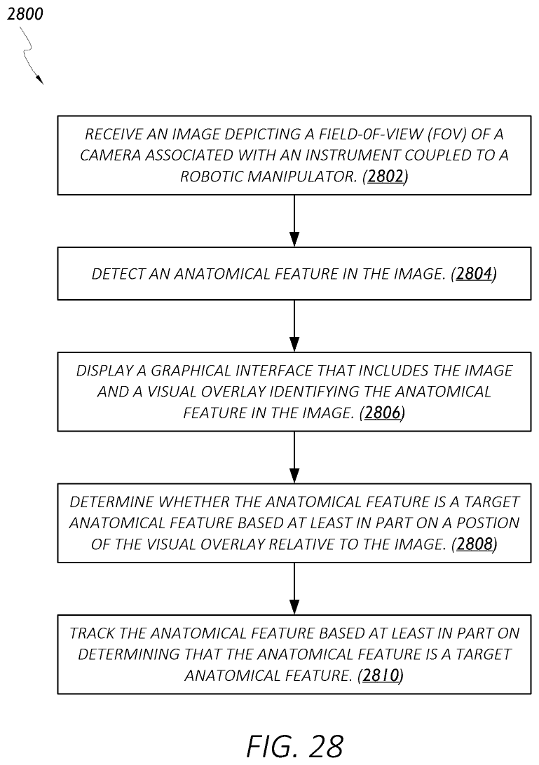

FIG. 28 shows an illustrative flowchart depicting an example target localization operation 2800, according to some implementations. In some implementations, the example operation 2800 may be performed by a controller for a robotic system such as the controller 2700 of FIG. 27.

The controller receives an image depicting a field-of-view (FOV) of a camera associated with an instrument coupled to a robotic manipulator (2802). The controller detects an anatomical feature in the image (2804). The controller displays a graphical interface that includes the image and a visual overlay identifying the anatomical feature in the image (2806). The controller determines whether the anatomical feature is a target anatomical feature based at least in part on a position of the visual overlay relative to the image (2808). The controller further tracks the anatomical feature based at least in part on determining that the anatomical feature is a target anatomical feature (2810).

### Additional Embodiments

Described herein are systems, devices, and methods to facilitate the identification, tracking, and targeting of various anatomical features based on certain sensor- and/or image-based position information, which may be obtained using, for example, an endoscope device or other medical instrument. Target anatomical feature localization in accordance with aspects of the present disclosure can facilitate the targeting of the anatomical feature(s) in connection with a medical procedure, such as a nephroscopy or other procedure accessing of the renal anatomy, for example.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A robotic system comprising:
a robotic manipulator configured to manipulate an instrument having a camera associated therewith; and
control circuitry communicatively coupled to the robotic manipulator, the control circuitry configured to:
receive an image depicting a field-of-view (FOV) of the camera associated with the instrument;
detect an anatomical feature in the image;
display a graphical interface that includes the image and a visual overlay indicating a location of the anatomical feature in the image;
determine whether the anatomical feature is a target anatomical feature based at least in part on a position of the visual overlay relative to the image; and
track the anatomical feature based at least in part on determining that the anatomical feature is a target anatomical feature.

2. The robotic system of claim 1, wherein the anatomical feature is detected based on a pretrained neural network.

3. The robotic system of claim 2, wherein the neural network is configured to detect the anatomical feature in the image based at least in part on position data indicating a position of the instrument, user input for controlling the instrument, or robotic command data that causes the robotic manipulator to manipulate the instrument.

4. The robotic system of claim 1, wherein the visual overlay comprises at least one of a bounding box, a binary mask, or an outline of the anatomical feature.

5. The robotic system of claim 4, wherein each pixel of the binary mask has a confidence value indicating a confidence in the pixel being correctly classified.

6. The robotic system of claim 1, wherein the determining of whether the anatomical feature is a target anatomical feature comprises determining whether the visual overlay remains centered in relation to the FOV of the camera for at least a threshold duration.

7. The robotic system of claim 1, wherein the determining of whether the anatomical feature is a target anatomical feature comprises:
receiving user input associated with a region of the graphical interface; and
determining whether the region coincides with the position of the visual overlay.

8. The robotic system of claim 1, wherein the determining of whether the anatomical feature is a target anatomical feature comprises:
receiving user input; and
determining whether the visual overlay is centered in the image responsive to receiving the user input.

9. The robotic system of claim 1, wherein the tracking of the anatomical feature comprises:
receiving user input via an input mechanism for controlling the robotic manipulator to manipulate the instrument;
determining whether the user input causes the instrument to move in a direction away from the anatomical feature; and
providing haptic feedback via the input mechanism responsive to determining that the user input causes the instrument to move in a direction away from the anatomical feature.

10. The robotic system of claim 1, wherein the tracking of the anatomical feature comprises preventing the robotic manipulator from manipulating the instrument in a direction away from the anatomical feature.

11. The robotic system of claim 1, wherein the tracking of the anatomical feature comprises displaying an indicator on the graphical interface directing a user to move the instrument in a direction of the anatomical feature.

12. The robotic system of claim 1, wherein the tracking of the anatomical feature comprises:
displaying a first reticle in the graphical interface that is centered on the visual overlay;
displaying a second reticle in the graphical interface that is centered in relation to the FOV of the camera; and
providing guidance via the graphical interface for manipulating the instrument so that the first reticle is aligned with the second reticle.

13. The robotic system of claim 1, wherein the tracking of the anatomical feature comprises:
causing the robotic manipulator to manipulate the instrument in a series of poses;
capturing, via the camera, a series of images associated with the series of poses, respectively;
detecting the anatomical feature in each image of the series of images; and
determining a three-dimensional position of the anatomical feature based on positions of the anatomical feature in each image of the series of images.

14. The robotic system of claim 13, wherein the control circuitry is further configured to provide guidance via the graphical interface for controlling the robotic manipulator to manipulate the instrument in the series of poses.

15. The robotic system of claim 14, wherein the guidance includes instructions to maintain the instrument in each pose of the series of poses for a duration associated with a respiration cycle.

16. The robotic system of claim 14, wherein the guidance includes a third-person point of view (POV) depicting the instrument in its current pose and further depicting the instrument in a next pose following the current pose in the series of poses.

17. The robotic system of claim 13, wherein the control circuitry causes the robotic manipulator to manipulate the instrument in the series of poses without user input.

18. The robotic system of claim 17, wherein the causing of the robotic manipulator to manipulate the instrument in the series of poses without user input comprises:
determining a difference between a current pose of the instrument and a next pose following the current pose in the series of poses; and
causing the robotic manipulator to manipulate the instrument to the next pose based on the difference between the current pose and the next pose.

19. A method of target localization, comprising:
receiving an image depicting a field-of-view (FOV) of a camera associated with an instrument coupled to a robotic manipulator;
detecting an anatomical feature in the image;
displaying a graphical interface that includes the image and a visual overlay identifying the anatomical feature in the image;
determining whether the anatomical feature is a target anatomical feature based at least in part on a position of the visual overlay relative to the image; and
tracking the anatomical feature based at least in part on determining that the anatomical feature is a target anatomical feature.

20. A controller for a robotic system, comprising:
a processing system; and
a memory storing instructions that, when executed by the processing system, cause the controller to:
receive an image depicting a field-of-view (FOV) of a camera associated with an instrument coupled to a robotic manipulator;
detect an anatomical feature in the image;
display a graphical interface that includes the image and a visual overlay identifying the anatomical feature in the image;
determine whether the anatomical feature is a target anatomical feature based at least in part on a position of the visual overlay relative to the image; and
track the anatomical feature based at least in part on determining that the anatomical feature is a target anatomical feature.
